(19) United States
(12) Patent Application Publication
(10) Pub. No.: US 2023/0212259 A1
(43) Pub. Date: Jul. 6, 2023

(54) YEAST DISPLAY LIBRARIES, ASSOCIATED COMPOSITIONS, AND ASSOCIATE METHODS OF USE

(71) Applicant: 3T BIOSCIENCES, INC., South San Francisco, CA (US)

(72) Inventors: David Victor Liu, Pacifica, CA (US); Hanspeter Gerber, San Carlos, CA (US); Leah Sibener, San Francisco, CA (US)

(21) Appl. No.: 17/800,993

(22) PCT Filed: Feb. 21, 2021
(86) PCT No.: PCT/US2021/018949

§ 371 Date: Aug. 19, 2022

Related U.S. Application Data
(60) Provisional application No. 62/980,088, filed on Feb. 21, 2020.

(51) Int. Cl. C07K 14/74; C12N 15/62; C12N 15/81

(52) U.S. Cl. CPC C07K 14/70539; C12N 15/81; C12N 15/625; C07K 2319/00; C12N 2800/102

(57) ABSTRACT

Described herein are single chain trimer (SCT) polypeptides comprising or consisting essentially of a target peptide, a first linker, at least a portion of a beta-2 microglobulin domain, a second linker, and at least a portion of a major histocompatibility complex (MHC) I alpha chain, or pharmaceutically acceptable derivatives thereof. The SCT polypeptides may further include a leader peptide, e.g., a PHO5, SUC2, app8, or HLA A2 leader sequence at the N-terminus of the target peptide. Further described herein are polypeptide compositions comprising or consisting essentially of a first polypeptide comprising a target peptide, and a second polypeptide comprising at least a portion of a beta-2 microglobulin domain, a second linker, and at least a portion of a major histocompatibility complex (MHC) I alpha chain, a third linker, and a tether peptide, or pharmaceutically acceptable derivatives thereof. The first polypeptide and/or the second polypeptide may further include a leader peptide, e.g., a PHO5, SUC2, app8, or HLA A2 leader sequence. The present disclosure also includes associated kits, methods, compositions, nucleotides, cells, and uses thereof.

### CROSS-REFERENCE TO RELATED APPLICATIONS

This application claims the benefit of priority to U.S. Provisional Application No. 62/980,088, filed on Feb. 21, 2020, which is incorporated herein by reference in its entirety.

### SEQUENCE LISTING

The instant application contains a Sequence Listing which has been submitted electronically in ASCII format and is hereby incorporated by reference in its entirety. The ASCII copy, created on Feb. 21, 2021, is named T105482_1060WO_SL_ST25.txt and is 31,760 bytes in size. The sequences listed are summarized in Table 1 below.

TABLE 1

Sequences Listed

| SEQ ID NO: | Brief Description of Sequence |
|---|---|
| 1 | Linker 1 with a {G2C} substitution |
| 2 | Linker 1 with {G2C, G4A} substitutions |
| 3 | Linker 1 with {G2C, G4A} substitutions |
| 4 | Linker 1 with a {G4A} substitution |
| 5 | NY-ESO-1 single chain trimer having a disulfide trapped linker at a Linker 1 position with a {G2C} substitution, and a MHC I alpha chain with a {Y84C} substitution |
| 6 | NY-ESO-1 single chain trimer having a disulfide trapped linker at a Linker 1 position with {G2C, G4A} substitutions, and a MHC I alpha chain with a {Y84C} substitution |
| 7 | NY-ESO-1 single chain trimer having a GGGAS linker at a Linker 1 position with a {G4A} substitution, and a MHC I alpha chain with a {Y84A} substitution |
| 8 | NY-ESO-1 single chain trimer in the absence of a Linker 1 |
| 9 | NY-ESO-1 single chain trimer having two amino acid residues in the C-terminal region of the NY-ESO-1 peptide, and a MHC I alpha chain with a {Y84C} substitution in the absence of a Linker 1 |
| 10 | Nucleotide sequence encoding a PHO5 leader peptide |
| 11 | Nucleotide sequence encoding a SUC2 leader peptide |
| 12 | Nucleotide sequence encoding an app8 leader peptide |
| 13 | Nucleotide sequence encoding an app8 EA leader peptide |
| 14 | Nucleotide sequence encoding a syn leader peptide |
| 15 | Nucleotide sequence encoding a syn EA leader peptide |
| 16 | Nucleotide sequence encoding an appWT leader peptide |
| 17 | Nucleotide sequence encoding an appWT EA leader peptide |
| 18 | Amino acid sequence of a PHO5 leader peptide |
| 19 | Amino acid sequence of a SUC2 leader peptide |
| 20 | Amino acid sequence of an app8 leader peptide |
| 21 | Amino acid sequence of an app EA leader peptide |
| 22 | Amino acid sequence of a syn leader peptide |
| 23 | Amino acid sequence of a syn EA leader peptide |
| 24 | Amino acid sequence of an appWT leader peptide |
| 25 | Amino acid sequence of an appWT EA leader peptide |

### BACKGROUND

T cells are the central mediators of adaptive immunity, through both direct effector functions and coordination and activation of other immune cells. Each T cell expresses a unique T cell receptor (TCR), selected for the ability to bind to major histocompatibility complex (MHC) molecules presenting peptides. TCR recognition of peptide-MHC (pMHC) drives T cell development, survival, and effector functions. The peptide-Major Histocompatibility Complex (pMHC) is a non-covalent complex of 3 proteins. In order to improve stability, the pMHC can be constructed as a single chain trimer (SCT), a single fusion protein with the general structure of P-L1-B-L2-A, where L1 and L2 are flexible linkers, P is a peptide ligand, and in the case of MHC Class I, A is a soluble form of the alpha chain of MHC I, and B is beta-2-microglobulin (Yu, 2002). In SCTs derived from MHC Class I, the Y84A mutation can be introduced into the MHC-alpha domain to better accommodate Linker 1 at the C terminus of the peptide ligand (Lybarger, 2003).

The SCT has been adapted for display on the surface of yeast for both MHC Class I and MHC Class II through the fusion to a yeast cell wall protein (e.g., Aga2) (Adams, 2011; Birnbaum, 2014; Gee, 2018). For MHC Class I, the yeast-displayed SCT has the general structure of P-L1-B-L2-A-L3-T, where T is a yeast cell wall protein (e.g., Aga2), L3 is a flexible linker, and P, B, A, L1 and L2 are as described previously. Peptide libraries in yeast-displayed SCT of MHC Class I and of Class II have enabled the de-orphanizing of a T cell receptor (TCR) through the identification of the cognate pMHC towards which the TCR is reactive, and identification of off-target cross reactivities to other pMHC (Birnbaum, 2014; Gee, 2018). In many cases, the off-target cross-reactive pMHCs are non-homologous to the intended pMHC target, suggesting that these libraries can more comprehensively identify reactive peptides than other methods that rely on sequence similarity.

Novel compositions and methods for the identification of T cell receptor ligands are needed.

### SUMMARY

Provided herein in certain embodiments are single chain trimer (SCT) polypeptides comprising or consisting essentially of a target peptide, a first linker, at least a portion of a beta-2 microglobulin domain, a second linker, and at least a portion of a major histocompatibility complex (MHC) I alpha chain, or pharmaceutically acceptable derivatives thereof.

In some aspects, the first linker is a peptide. In some aspects, the first linker has an amino acid sequence that is at least about 70% homologous to GCGGSGGGGSGGGGS (SEQ ID NO: 1), at least about 80% homologous to GCGGSGGGGSGGGGS (SEQ ID NO: 1), at least about 85% homologous to GCGGSGGGGSGGGGS (SEQ ID NO: 1), at least about 90% homologous to GCGGSGGGGSGGGGS (SEQ ID NO: 1), at least about 95% homologous to GCGGSGGGGSGGGGS (SEQ ID NO: 1), at least about 97.5% homologous to GCGGSGGGGSGGGGS (SEQ ID NO: 1), or at least about 99% homologous to GCGGSGGGGSGGGGS (SEQ ID NO: 1). In some aspects, the first linker has an amino acid sequence that is GCGGSGGGGSGGGGS (SEQ ID NO: 1).

In some aspects, the first linker has an amino acid sequence that is at least about 70% homologous to GCGASGGGGSGGGGSGGGGS (SEQ ID NO: 2), at least about 80% homologous to GCGASGGGGSGGGGSGGGGS (SEQ ID NO: 2), at least about 85% homologous to GCGASGGGGSGGGGSGGGGS (SEQ ID NO: 2), at least about 90% homologous to GCGASGGGGSGGGGSGGGGS (SEQ ID NO: 2), at least about 95% homologous to GCGASGGGGSGGGGSGGGGS (SEQ ID NO: 2), at least about 97.5% homologous to GCGASGGGGSGGGGSGGGGS (SEQ ID NO: 2), or at least about 99% homologous to GCGASGGGGSGGGGSGGGGS (SEQ ID NO: 2). In some aspects, the first linker has an amino acid sequence that is GCGASGGGGSGGGGSGGGGS (SEQ ID NO: 2).

In some aspects, the first linker has an amino acid sequence that is at least about 70% homologous to GCGASGGGGSGGGGS (SEQ ID NO: 3), at least about 80% homologous to GCGASGGGGSGGGGS (SEQ ID NO: 3), at least about 85% homologous to GCGASGGGGSGGGGS (SEQ ID NO: 3), at least about 90% homologous to GCGASGGGGSGGGGS (SEQ ID NO: 3), at least about 95% homologous to GCGASGGGGSGGGGS (SEQ ID NO: 3), at least about 97.5% homologous to GCGASGGGGSGGGGS (SEQ ID NO: 3), or at least about 99% homologous to GCGASGGGGSGGGGS (SEQ ID NO: 3). In some aspects, the first linker has an amino acid sequence that is GCGASGGGGSGGGGS (SEQ ID NO: 3).

In some aspects, at least the portion of the MHC I alpha chain comprises an amino acid substitution compared to a wild-type MHC I alpha chain. In some aspects, the amino acid substitution is {Y84C}. In some aspects, the second amino acid counted from the N-terminus of the first linker is C. In some aspects, the first linker has an amino acid substitution {G2C}. In some aspects, a disulfide bridge forms between the first linker and the MHC I alpha chain. In some aspects, the disulfide bridge forms at (i) the {G2C} of the first linker, or the second amino acid counted from the N-terminus of the first linker, wherein the amino acid is C, and (ii) the {Y84C} of the MHC I alpha chain.

In some aspects, the amino acid substitution in the portion of the MHC I alpha chain is {Y84A}.

In other aspects, the first linker has an amino acid sequence that is at least about 70% homologous to GGGASGGGGSGGGGS (SEQ ID NO: 4), at least about 80% homologous to GGGASGGGGSGGGGS (SEQ ID NO: 4), at least about 85% homologous to GGGASGGGGSGGGGS (SEQ ID NO: 4), at least about 90% homologous to GGGASGGGGSGGGGS (SEQ ID NO: 4), at least about 95% homologous to GGGASGGGGSGGGGS (SEQ ID NO: 4), at least about 97.5% homologous to GGGASGGGGSGGGGS (SEQ ID NO: 4), or at least about 99% homologous to GGGASGGGGSGGGGS (SEQ ID NO: 4). In some aspects, the first linker has an amino acid sequence that is GGGASGGGGSGGGGS (SEQ ID NO: 4).

In some aspects, the SCT polypeptides comprise or consist essentially of a tag, a third linker, and/or a tether peptide. In some aspects, the tether peptide is Aga2.

In some aspects, the SCT polypeptides comprise or consist essentially of a leader peptide. In some aspects, the leader peptide is located at the N-terminus of the target peptide. In some aspects, the leader peptide directs the SCT polypeptides to the ER, facilitates ER to Golgi transport, and/or facilitates aspects of late secretory processing. Leader sequences that may be used in the present disclosure include, but are not limited to, the Aga2 leader sequence, the MFα-1 pre-pro secretory sequence, the HLA A2 leader sequences, PHO5 (SEQ ID NO: 18), SUC2 (SEQ ID NO: 19), app8 (SEQ ID NO: 20), app8 EA (SEQ ID NO: 21), syn (SEQ ID NO: 22), syn EA (SEQ ID NO: 23), appWT (SEQ ID NO: 24), appWT EA (SEQ ID NO: 25), and variants thereof.

In some aspects of the disclosed SCT polypeptides, the leader peptide shares 70% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 80% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 85% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 90% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 95% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 97.5% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 99% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide comprises a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some preferred aspects, the leader peptide comprises a sequence that shares 100% sequence identity with PHO5 (SEQ ID NO: 18) or SUC2 (SEQ ID NO: 19), or consists essentially of a sequence of PHO5 (SEQ ID NO: 18) or SUC2 (SEQ ID NO: 19).

In additional aspects of the disclosed SCT polypeptides, the leader sequence functions essentially as a sequence of PHO5 (SEQ ID NO: 18) or SUC2 (SEQ ID NO: 19), e.g., with similar efficiency in directing SCT polypeptides to the ER, facilitating ER to Golgi transport, and/or facilitating aspects of late secretory processing.

Also provided herein in certain aspects are polypeptide compositions comprising or consisting essentially of a first polypeptide comprising a target peptide, and a second polypeptide comprising at least a portion of a beta-2 microglobulin domain, a second linker, and at least a portion of a major histocompatibility complex (MHC) I alpha chain, a third linker, and a tether peptide, or pharmaceutically acceptable derivatives thereof.

In some aspects, the first polypeptide and the second polypeptide each further comprise a leader sequence, such as the Aga2 leader sequence, the MFα-1 pre-pro secretory sequence, the HLA A2 leader sequences, PHO5 (SEQ ID NO: 18), SUC2 (SEQ ID NO: 19), app8 (SEQ ID NO: 20), app8 EA (SEQ ID NO: 21), syn (SEQ ID NO: 22), syn EA (SEQ ID NO: 23), appWT (SEQ ID NO: 24), appWT EA (SEQ ID NO: 25), and variants thereof as described herein. Nucleotides encoding the first polypeptide and the second polypeptide may be further contained in a vector or in separate vectors.

In specific aspects, the leader sequence of the first polypeptide and/or the leader sequence of the second polypeptide share(s) 70% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23); 80% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23); 85% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23); 90% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23); 95% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23); 97.5% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23); or 99% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader sequence of the first polypeptide and/or the leader sequence of the second polypeptide comprise(s) a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some preferred aspects, the leader sequence of the first polypeptide and/or the leader sequence of the second polypeptide comprise(s) a sequence that is 100% identical to PHO5 (SEQ ID NO: 18) or SUC2 (SEQ ID NO: 19), or that consists essentially of the sequence of PHO5 (SEQ ID NO: 18) or SUC2 (SEQ ID NO: 19).

In other aspects, the first polypeptide further comprises a peptide fragment. In some aspects, the peptide fragment comprises at least two amino acids. In some aspects, the at least two amino acids are G and C. In some aspects, at least the portion of the MHC I alpha chain comprises an amino acid substitution compared to a wild-type MHC I alpha chain. In some aspects, the amino acid substitution is {Y84C}. In some aspects, a disulfide bridge forms between the peptide fragment and the MHC I alpha chain. In these aspects, the disulfide bridge forms at between the C amino acid of the peptide fragment and the {Y84C} of the MHC I alpha chain. In some aspects, the amino acid substitution of the portion of the MHC I alpha chain comprises an amino acid substitution compared to a wild-type MHC I alpha chain is {Y84A}.

Also provided herein in certain aspects are libraries of polypeptides comprising or consisting essentially of at least one of the SCT polypeptides of the present disclosure and/or at least one of the polypeptide compositions of the present disclosure. The disclosed libraries can also comprise or consist essentially of two or more the SCT polypeptide or two or more of the polypeptide compositions of as described herein.

Further provided herein in certain aspects are pharmaceutical compositions comprising or consisting essentially of at least one of the SCT polypeptides of the present disclosure and/or at least one of the polypeptide compositions of the present disclosure.

Also provided herein in certain aspects are cells comprising or consisting essentially of at least one of the SCT polypeptides of the present disclosure and/or at least one of the polypeptide compositions of the present disclosure. In some aspects, expression of the SCT polypeptides or the polypeptide compositions is inducible in the cells. In some aspects, the cells are yeast cells, e.g., Saccharomyces cerevisiae cells.

Further provided herein in certain aspects are first nucleic acids comprising or consisting essentially of a second nucleic acid encoding at least one of the SCT polypeptides of the present disclosure and/or at least one of the polypeptide compositions of the present disclosure.

Also provided herein in certain aspects are expression vectors comprising or consisting essentially of at least one of the nucleic acids of the present disclosure. In some aspects, expression of the SCT polypeptides and/or the polypeptide compositions of the present disclosure is inducible in in the vector or in the cells.

Further provided herein in certain aspects are kits comprising or consisting essentially of a first container comprising the pharmaceutical compositions of the present disclosure in solution or in lyophilized form. The kits optionally comprise a second container containing a diluent or reconstituting solution for the lyophilized formulation and/or instructions for (i) use of the solution or (ii) reconstitution and/or use of the lyophilized composition form.

Also provided herein in certain aspects are methods comprising or consisting essentially of preparing one or more polypeptides selected from the group consisting of the SCT polypeptides of the present disclosure and the polypeptide compositions of the present disclosure, the method comprising co-expressing protein disulfide isomerase with one or more of the polypeptides of the present disclosure in cells, culturing the cells, and isolating the one or more polypeptides from the cell or a culture medium thereof. In some aspects, the cells are yeast cells, e.g., Saccharomyces cerevisiae cells.

Further provided herein in certain aspects are methods of displaying a target peptide on a cell surface, the method comprising modifying the cells with the nucleic acids of the present disclosure comprising, consisting essentially of, or encoding at least one of the SCT polypeptides of the present disclosure and/or at least one of the polypeptide compositions of the present disclosure. In some aspects, the methods optionally comprise inducing expression of the SCT polypeptides or the polypeptide compositions of the present disclosure in the cells. In some aspects, the cells are yeast cells, e.g., Saccharomyces cerevisiae cells.

Also provided herein in certain aspects are in vitro methods for producing activated T cells, comprising or consisting essentially of contacting T cells with one or more of the SCT polypeptides of the present disclosure and/or one or more of the polypeptide compositions of the present disclosure.

Further provided herein in certain aspects are activated T cells, produced by the methods of the present disclosure. In some aspects, the activated T cells selectively recognize a cell expressing one or more peptides selected from the group consisting of the target peptides of the present disclosure.

### BRIEF DESCRIPTION OF THE DRAWINGS

FIGS. 1A and 1B are illustrations of an SCT having a disulfide trapped linker (FIG. 1A; “dt-SCT”) and an alanine linker (FIG. 1B; “GGGAS-Linker”) in accordance with embodiments of the present technology.

FIGS. 2A and 2B are annotated sequences of NY-ESO-1 SCT in accordance with embodiments of the present technology. FIG. 2A includes a disulfide trapped linker at a Linker 1 position with a {G2C} substitution, and a {Y84C} substitution in a MHC I alpha chain in accordance with embodiments of the present technology (SEQ ID NO: 5). FIG. 2B includes a disulfide trapped linker at a Linker 1 position with {G2C, G4A} substitutions, and a {Y84C} substitution in a MHC I alpha chain in accordance with embodiments of the present technology (SEQ ID NO: 6).

FIG. 3 is an annotated sequence of NY-ESO-1 SCT having a GGGAS linker at a Linker 1 position with a {G4A} substitution, and a MHC I alpha chain with a {Y84A} substitution in accordance with embodiments of the present technology (SEQ ID NO: 7).

FIG. 4 is an illustration of a secreted peptide for HLA capture in accordance with embodiments of the present technology.

FIG. 5 is an illustration of a method for using the secreted peptide for HLA capture of FIG. 4 in accordance with embodiments of the present technology.

FIG. 6 is an illustration of another method for using the secreted peptide for HLA capture of FIGS. 4 and 5 in accordance with embodiments of the present technology.

FIG. 7 is an annotated sequence of NY-ESO-1 peptide-MHC in the absence of a Linker 1 in accordance with embodiments of the present technology (SEQ ID NO: 8).

FIG. 8 is an annotated sequence of NY-ESO-1 peptide-MHC having two amino acid residues on the C-terminal region of the NY-ESO-1 peptide and a MHC I alpha chain with a {Y84C} substitution in the absence of a Linker 1 (SEQ ID NO: 9) in accordance with embodiments of the present technology.

FIG. 9 is a set of graphs showing effect of Linker 1 in TCR binding on an SCT. Empty HLA A2 yeast were pulsed with a peptide, and stained with TCR tetramer and streptavidin-phycoerythrin (SA-PE). Histograms are gated on FLAG-FITC fluorescence intensity (x axis). Y axis shows mean fluorescence intensity for PE. Histograms show binding of TCR tetramers (AFP, DMF5, 1G4LY, UQK, and MAGEA4, respectively) to AFP, MART1, NY-ESO-9V, NY-ESO-9C, and MAGE-A4 peptide, respectively, with or without Linker 1, bound to A2 yeast. “-L1” indicates an SCT without Linker 1. “DMSO” indicates no peptide control.

FIG. 10 is a set of graphs showing effect of Linker 1 on TCR binding to an SCT on yeast clones and empty A2 yeast pulsed with peptides. FIG. 10A is a chart showing MART1 peptide expression (x axis) and DMF5 TCR tetramer binding (y axis) in clonal MART1-displaying yeast. FIG. 10B is a histogram showing binding of DMF5 TCR tetramer to empty A2 yeast pulsed with MART1 peptide with or without Linker 1, or no peptide. FIGS. 10C and 10D are charts showing expression of MART1 SCT and NY-ESO-9V dt-SCT, respectively, (x axis) and c58c61 TCR monomer binding (y axis) in clonal yeast. FIG. 11E is a histogram showing binding of c58c61 TCR monomer to empty A2 yeast pulsed with NY-ESO-9C peptide or NY-ESO-9V peptide with or without Linker 1, or no peptide control.

FIG. 11 is a set of illustrations of a method for HLA capture in accordance with embodiments of the present technology. FIG. 11A shows a method for HLA capture by pulsing empty A2 yeast with HLA peptides. FIG. 11B shows a method for HLA capture using secreted peptide from clonal yeast expressing SCT.

FIG. 12 is a set of charts showing HLA display (x axis) and TCR tetramer binding (y axis) in empty A2 yeast pulsed with peptides (top row) or in yeast clones expressing SCTs (bottom row) stained with the respective TCR tetramer. In the first column, ** indicates that the empty A2 yeast were pulsed with NY-ESO-9V; and * indicates clonal yeast expressing NY-ESO dt-SCT. In the second to the sixth column, from left to right, peptide pulsed (top) or contained in the SCTs (bottom) were MART-1, AFP, AFP, MAGE-A4, and MAGE-A4, respectively. 1G4LY, DMF5, AFP-1, AFP-2, MAGE-A4-1, and MAGE-A4-2 indicate TCR tetramers.

FIG. 13A is an illustration of the MFα-1 pre-pro secretory sequence, which is used for heterologous protein expression in yeast. FIG. 13B is an illustration of SCT constructs with a leader sequence in accordance with aspects of the present technology. In some aspects, the leader sequence comprises Aga2, PHO5, SUC2, app8, or HLA A2 leader sequence.

FIG. 14A is a set of charts showing pHLA display (y axis) and TCR tetramer binding (x axis) in clonal yeast induced to express SCTs having a NY-ESO-9V peptide and a pre-pro secretory sequence appWT, appWT EA, app8, or app8 EA (NY-ESO-9V A2 appWT, NY-ESO-9V A2 appWT EA, NY-ESO-9V A2 app8, NY-ESO-9V A2 app8 EA). FIG. 14B is a set of charts showing pHLA display (y axis) and TCR tetramer binding (x axis) in clonal yeast induced to express SCTs having a NY-ESO-9V peptide and a pre-pro secretory sequence syn, or syn EA (NY-ESO-9V A2 syn, NY-ESO-9V A2 syn EA). Columns c5c1, c58c61, and 1G4-LY indicate TCR tetramers. The fourth and fifth columns (“SAPE, FLAG-FITC only” and “SAPE only”) indicates negative controls.

FIG. 15 is a set of charts showing pHLA display (y axis) and TCR tetramer binding (x axis) in clonal yeast induced to express SCTs having a NY-ESO-9V peptide and a PHO5 leader sequence, a SUC2 leader sequence, or a GGGAS linker (NY-ESO-9V A2 PHO5, NY-ESO-9V A2 SUC2, NY-ESO-9V A2 GGGAS). Columns c5c1, c58c61, and 1G4-LY indicate TCR tetramers. The fourth and fifth columns (“SAPE, FLAG-FITC only” and “SAPE only”) indicates negative controls.

FIG. 16A is a graph showing TCR tetramer binding (y axis) in clonal yeast induced to express SCTs described in FIGS. 14-15 (x axis). FIG. 16B is a graph showing pHLA display (y axis) in the same set of yeast as described in FIG. 16A.

FIG. 17 is a set of charts showing pHLA display (y axis) and TCR tetramer binding (x axis) in clonal yeast induced to express the following SCTs, respectively: PHOS-NY-ESO (having a PHO5 leader sequence and a NY-ESO peptide), SUC2-NY-ESO (having a SUC2 leader sequence and a NY-ESO peptide), PHO5-MART-1 (having a PHO5 leader sequence and a MART-1 peptide), SUC2-MART-1 (having a SUC2 leader sequence and a MART-1 peptide), PHO5-MART-1-cyclic (having a PHO5 leader sequence and a MART-1-cyclic peptide), and SUC2-MART-1-cyclic (having a SUC2 leader sequence and a MART-1-cyclic peptide). Columns “no stain” and “SAPE / FLAG-FITC” are negative controls. Tetramers c58c61 and DMF5 indicate TCR tetramers.

FIG. 18 is a set of charts showing pHLA display (y axis) and TCR tetramer binding (x axis) in clonal yeast induced to express the following SCTs, respectively: PHOS-NY-ESO (having a PHO5 leader sequence and a NY-ESO peptide), SUC2-NY-ESO (having a SUC2 leader sequence and a NY-ESO peptide), PHO5-AFP (having a PHO5 leader sequence and a AFP peptide), SUC2-AFP (having a SUC2 leader sequence and a AFP peptide), PHOS-MAGE-A4 (having a PHO5 leader sequence and a MAGE-A4 peptide), and SUC2-MAGE-A4 (having a SUC2 leader sequence and a MAGE-A4 peptide). Columns “no stain” and “SAPE / FLAG-FITC” are negative controls. Tetramers c58c61, AFP1, AFP2, and MAGE-A4 indicate TCR tetramers.

FIG. 19A is a graph showing TCR tetramer binding (y axis) in clonal yeast induced to express SCTs described in FIGS. 17 and 18 (x axis). FIG. 19B is a graph showing pHLA display (y axis) in clonal yeast induced to express SCTs described in FIGS. 17 and 18 (x axis).

### DETAILED DESCRIPTION

Identification of TCRs and cognate antigens provides therapeutic strategies for immunotherapy, including screening of patient T cells for responsiveness, vaccination with synthetic peptide fragments of the cognate antigens or nucleic acids encoding linkers, cell-based therapies, protein-based therapies, etc. Unlike other approaches to identify potential TCRs, the present disclosure provides second-generation polypeptides having (a) at least one linker (e.g., a second-generation linker) that (i) includes a disulfide bridge between at least one amino acid residue of the linker and at least one amino acid residue within an MHC domain (e.g., a disulfide trapped single chain trimer (“dt-SCT”)), or (ii) an alanine residue at amino acid residue 4 (e.g., a “GGGAS Linker”), or (b) a secreted peptide and an MHC polypeptide having at least one linker. In the secreted approach, the secreted peptide can optionally include two amino acids that form a disulfide bridge with the MHC polypeptide. In either format, the disulfide bridge may increase binding to potential TCRs. Accordingly, the present disclosure includes second-generation polypeptides having second-generation linkers and/or secreted peptides, associated libraries, polypeptides, compositions, kits, cells, methods of preparing, and methods of using the same. The second-generation polypeptides, associated libraries, polypeptides, compositions, kits, cells, methods of preparing, and methods of using the same disclosed herein are useful in identifying novel TCRs that may be useful for treating a disease and/or a condition in a subject.

The second-generation polypeptides of the present disclosure differ from polypeptides having other linkers (e.g., first-generation polypeptides), such as first-generation linkers, in several ways. Second-generation linkers of the present disclosure include at least one cysteine residue or at least one alanine residue whereas first generation linkers include at least one glycine and at least one serine residue. In addition, second-generation linkers of the present disclosure optionally include at least one disulfide bridge whereas the first-generation linkers do not. Still further, the second-generation polypeptides of the present disclosure can also include a second-generation linker-free design, such as a secreted peptide which optionally includes two amino acid residues that can form a disulfide bridge with the MHC polypeptide.

The second-generation polypeptide and libraries of the present disclosure are also improved as compared to existing polypeptides and libraries by incorporation of a leader sequence that enables improved presentation of target peptides.

In certain aspects disclosed herein, the second-generation polypeptides and libraries include second-generation linkers and the specific leader sequences showing improved presentation of target peptides.

The following issued patent and patent application publications are herein incorporated by reference as if each individual issued patent and patent application publication was specifically and individually indicated to be incorporated by reference in its entirety: U.S. Pat. No. U.S. 8,450,247, Peelle et al.; U.S. Pat. Publication No. 2010/0210473, Bowley et al.; U.S. Pat. Publication No. 2004/0146976, Dane et al.; International Patent Publication No. WO 2004/015395; International Patent Publication No. WO 2005/116646; International Patent Publication No. WO 2012/022975; and U.S. Patent Publication No. 2017/0192011, Birnbaum et al.

Reference throughout this specification to “one example,” “an example,” “one embodiment,” “an embodiment,” “one aspect,” or “an aspect” means that a particular feature, structure, or characteristic described in connection with the example is included in at least one example of the present disclosure. Thus, the occurrences of the phrases “in one example,” “in an example,” “one embodiment,” “an embodiment,” “one aspect,” or “an aspect” in various places throughout this specification are not necessarily all referring to the same example, embodiment, and/or aspect.

The headings provided herein are for convenience only and are not intended to limit or interpret the scope or meaning of the present disclosure.

The following description of the present disclosure is merely intended to illustrate various embodiments of the present disclosure. As such, the specific modifications discussed herein are not to be construed as limitations on the scope of the present disclosure. It will be apparent to one skilled in the art that various equivalents, changes, and modifications may be made without departing from the scope of the present disclosure, and it is understood that such equivalent embodiments are to be included herein.

### I. Definitions

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein is to be understood to include any integer within the recited range, unless otherwise indicated. As used herein, the term “about” means ± 20% of the indicated range, value, or structure, unless otherwise indicated. It should be understood that the terms “a” and “an” as used herein refer to “one or more” of the enumerated regions. Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense that one having skill in the art would understand the convention (e.g., “a system having at least one of A, B, and C” would include, but not be limited to, systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense that one having skill in the art would understand the convention (e.g., “a system having at least one of A, B, or C” would include, but not be limited to, systems that have A alone, B alone, C alone, A and B together, A and C together, B and C together, and/or A, B, and C together, etc.). As used herein, the terms “include,” “have,” and “comprise” are used synonymously, which terms and variants thereof are intended to be construed as non-limiting. Further, headings provided herein are for convenience only and do not interpret the scope or meaning of the claimed embodiments.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

The terms “treat,” “treating,” and “treatment” as used herein with regard to solid cancers refers to partial or total inhibition of tumor growth, reduction of tumor size, complete or partial tumor eradication, reduction or prevention of malignant growth, partial or total eradication of cancer cells, or some combination thereof. The terms “patient” and “subject” are used interchangeably herein.

A “subject in need thereof” as used herein refers to a mammalian subject, preferably a human, who has been diagnosed with cancer, is suspected of having cancer, and/or exhibits one or more symptoms associated with cancer.

The term “major histocompatibility complex” (MHC) proteins (also called human leukocyte antigens, HLA, or the H2 locus in the mouse) are protein molecules expressed on the surface of cells that confer a unique antigenic identity to these cells. MHC/HLA antigens are target molecules that are recognized by T-cells and natural killer (NK) cells as being derived from the same source of hematopoietic reconstituting stem cells as the immune effector cells (“self”) or as being derived from another source of hematopoietic reconstituting cells (“non-self”). Two main classes of HLA antigens are recognized: HLA class I and HLA class II. MHC proteins as used herein includes MHC proteins from any mammalian or avian species, e.g. primate sp., particularly humans; rodents, including mice, rats and hamsters; rabbits; equines, bovines, canines, felines, etc. Of particular interest are the human HLA proteins, and the murine H-2 proteins. Included in the HLA proteins are the class II subunits HLA-DPα, HLA-DPβ, HLA-DQα, HLA-DQβ, HLA-DRα and HLA-DRβ, and the class I proteins HLA-A, HLA-B, HLA-C, and β2-microglobulin. Included in the murine H-2 subunits are the class I H-2K, H-2D, H-2L, and the class II I-Aα, I-Aβ, I-Eα and I-Eβ, and β2-microglobulin.

As used herein, the term “class II HLA/MHC” binding domains comprise the α1 and α2 domains for the α chain, and the β1 and β2 domains for the β chain. Not more than about 10, usually not more than about 5, preferably none of the amino acids of the transmembrane domain will be included. The deletion will be such that it does not interfere with the ability of the α2 or β2 domain to bind peptide ligands. Class II HLA/MHC binding domains also refers to the binding domains of a major histocompatibility complex protein that are soluble domains of Class II α and β chain. Class II HLA/MHC binding domains include domains that have been subjected to mutagenesis and selected for amino acid changes that enhance the solubility of the single chain polypeptide, without altering the peptide binding contacts.

As used herein, the term “class I HLA/MHC” binding domains includes the α1, α2 and α3 domain of a Class I allele, including without limitation HLA-A, HLA-B, HLA-C, H-2K, H-2D, H-2L, which are combined with β2-microglobulin. Not more than about 10, usually not more than about 5, preferably none of the amino acids of the transmembrane domain will be included. The deletion will be such that it does not interfere with the ability of the domains to bind peptide ligands.

The “MHC binding domains”, as used herein, refers to a soluble form of the normally membrane-bound protein. The soluble form is derived from the native form by deletion of the transmembrane domain. The MHC binding domain protein is truncated, removing both the cytoplasmic and transmembrane domains and includes soluble domains of Class II alpha and beta chain. “MHC binding domains” also refers to binding domains that have been subjected to mutagenesis and selected for amino acid changes that enhance the solubility of the single chain polypeptide, without altering the peptide binding contacts.

“MHC context” as used herein refers to an interaction being in the presence of an MHC with non-covalent interactions with the MHC and an antigen. The function of MHC molecules is to bind peptide fragments derived from pathogens and display them on the cell surface for recognition by the appropriate T cells. Thus, TCR recognition can be influenced by the MHC protein that is presenting the antigen. The term MHC context refers to the recognition by a TCR of a given peptide, when it is presented by a specific MHC protein.

A “library” of second-generation polypeptides (also referred to herein as “polypeptides”), or of nucleic acids encoding such polypeptides, having the formula P-L₁-β-L₂-α, P-L₁-β-L₂-α-L₃-T, β-L₂-α, or β-L₂-α-L₃-T. In the library of polypeptides for polypeptides having L1, L1 is a disulfide trapped linker having the amino acid sequence GCGASGGGGSGGGGSGGGGS (SEQ ID NO: 2) or GCGASGGGGSGGGGS (SEQ ID NO: 3) (“dt-SCT”), or a GGGAS N-terminal linker having the amino acid sequence GGGASGGGGSGGGGS (SEQ ID NO: 4) (“GGGAS-Linker 1”). Without wishing to be bound by theory, GGGAS-Linker 1 has been shown support TCR binding to 1G4 and its variants in mammalian cells (Zhao, 2007). In some embodiments, L1 of the dt-SCT can optionally have the sequence GCGGSGGGGSGGGGS (SEQ ID NO: 1). L2 and L3 are each flexible linkers of from about 4 to about 20 amino acids in length, e.g. comprising glycine, serine, alanine, etc., α is a soluble form of at least a portion of a domain of a class I MHC protein or at least a portion of a domain of class II a MHC protein; β is a soluble form of (i) a β chain of a class II MHC protein or (ii) β2 microglobulin of a class I MHC protein; T is a domain that allows the polypeptide to be tethered to a cell surface, including without limitation yeast Aga2; and P is a peptide ligand. The library of polypeptides includes at least 10⁶, at least 10⁷, at least 10⁸, at least 10⁹, or at least 10¹⁰ different polypeptides having at least one of the formulas described herein.

An “allele” is one of the different nucleic acid sequences of a gene at a particular locus on a chromosome. One or more genetic differences can constitute an allele. An important aspect of the HLA gene system is its polymorphism. Each gene, MHC class I (A, B and C) and MHC class II (DP, DQ and DR) exists in different alleles. Current nomenclature for HLA alleles are designated by numbers, as described by Marsh et al.: Nomenclature for factors of the HLA system, 2010. Tissue Antigens 75:291-455, herein specifically incorporated by reference. For HLA protein and nucleic acid sequences, see Robinson et al. (2011), the IMGT/HLA database, Nucleic Acids Research 39 Suppl 1:D1171-6, herein specifically incorporated by reference.

“T cell receptor” (TCR), refers to an antigen/MHC binding heterodimeric protein product of a vertebrate (e.g., mammalian, TCR gene complex, including the human TCR α, β, γ, and δ chains). For example, the complete sequence of the human β TCR locus has been sequenced, as published by Rowen 1996; the human TCR locus has been sequenced and resequenced, for example, see Mackelprang 2006; see a general analysis of the T-cell receptor variable gene segment families in Arden 1995; each of which is herein specifically incorporated by reference for the sequence information provided and referenced in the publication.

The terms “recipient,” “individual,” “subject,” “host,” and “patient” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

The terms “peptide,” “polypeptide,” and “protein” are used interchangeably to refer to a polymer of amino acid residues, and are not limited to a minimum length, though a number of amino acid residues may be specified (e.g., 9mer is nine amino acid residues). Polypeptides may include amino acid residues including natural and/or non-natural amino acid residues. Polypeptides may also include fusion proteins. The terms also include post-expression modifications of the polypeptide, for example, glycosylation, sialylation, acetylation, phosphorylation, and the like. In some embodiments, the polypeptides may contain modifications with respect to a native or natural sequence, as long as the protein maintains the desired activity. These modifications may be deliberate, such as through site-directed mutagenesis, or may be accidental, such as through mutations of hosts which produce the proteins or errors due to PCR amplification.

The term “acidic residue” refers to amino acid residues in D- or L-form having sidechains comprising acidic groups. Exemplary acidic residues include D and E.

The term “amide residue” refers to amino acids in D- or L-form having sidechains comprising amide derivatives of acidic groups. Exemplary residues include N and Q.

The term “aromatic residue” refers to amino acid residues in D- or L-form having sidechains comprising aromatic groups. Exemplary aromatic residues include F, Y, and W.

The term “basic residue” refers to amino acid residues in D- or L-form having sidechains comprising basic groups. Exemplary basic residues include H, K, and R.

The term “hydrophilic residue” refers to amino acid residues in D- or L-form having sidechains comprising polar groups. Exemplary hydrophilic residues include C, S, T, N, and Q.

The term “nonfunctional residue” refers to amino acid residues in D- or L-form having sidechains that lack acidic, basic, or aromatic groups. Exemplary nonfunctional amino acid residues include M, G, A, V, I, L, and norleucine (Nle).

The term “neutral hydrophobic residue” refers to amino acid residues in D- or L-form having sidechains that lack basic, acidic, or polar groups. Exemplary neutral hydrophobic amino acid residues include A, V, L, I, P, W, M, and F.

The term “polar hydrophobic residue” refers to amino acid residues in D- or L-form having sidechains comprising polar groups. Exemplary polar hydrophobic amino acid residues include T, G, S, Y, C, Q, and N.

The term “hydrophobic residue” refers to amino acid residues in D- or L-form having sidechains that lack basic or acidic groups. Exemplary hydrophobic amino acid residues include A, V, L, I, P, W, M, F, T, G, S, Y, C, Q, and N.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or 1), Leucine (Leu or L), Methionine (Met or M), Valine (Val or V); and Group 6: Phenylalanine (Phe or F), Tyrosine (Tyr or Y), Tryptophan (Trp or W). Additionally, or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include sulfur-containing: Met and Cysteine (Cys or C); acidic: Asp, Glu, Asn, and Gln; small aliphatic, nonpolar, or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company. Variant proteins, peptides, polypeptides, and amino acid sequences of the present disclosure can, in certain embodiments, comprise one or more conservative substitutions relative to a reference amino acid sequence.

“Nucleic acid molecule” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and polydeoxyribonucleic acid (DNA), which includes cDNA, genomic DNA, and synthetic DNA, either of which may be single or doublestranded. A nucleic acid molecule encoding an amino acid sequence includes all nucleotide sequences that encode the same amino acid sequence.

“Percent (%) sequence identity” with respect to a reference polypeptide sequence is the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN, or Megalign (DNASTAR) software, or other software appropriate for nucleic acid sequences. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a some % amino acid sequence identity to, with, or against a given amino acid sequence B) is calculated as follows: 100 times the fraction X/Y, where X is the number of amino acid residues scored as identical matches by the sequence alignment program ALIGN-2 in that program’s alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “isolated” means that the material is removed from its original environment (e.g., the natural environment if it is naturally occurring). Such nucleic acid could be part of a vector and/or such nucleic acid or polypeptide could be part of a composition (e.g., a cell lysate), and still be isolated in that such vector or composition is not part of the natural environment for the nucleic acid or polypeptide.

As used herein, the terms “homologous,” “homology,” or “percent homology” when used herein to describe a nucleic acid sequence relative to a reference sequence, can be determined using the formula described by Karlin & Altschul 1990, modified as in Karlin & Altschul 1993. Such a formula is incorporated into the basic local alignment search tool (BLAST) programs of Altschul 1990. Percent homology of sequences can be determined using the most recent version of BLAST, as of the filing date of this application. Homologous sequences described herein include sequences having the same percentage identity as the indicated percentage homology. Sequences sharing a percentage identity are understood in the art to mean those sequences sharing the indicated percentage of same residues over the length of the reference sequence (e.g., the linker or leader sequences disclosed herein and in the sequence listing).

A “functional variant” refers to a polypeptide or polynucleotide that is structurally similar or substantially structurally similar to a parent or reference compound of this disclosure, but differs, in some contexts slightly, in composition (e.g., one base, atom, or functional group is different, added, or removed; or one or more amino acids are substituted, mutated, inserted, or deleted), such that the polypeptide or encoded polypeptide is capable of performing at least one function of the encoded parent polypeptide with at least 50% efficiency of activity of the parent polypeptide.

As used herein, a “functional portion” or “functional fragment” refers to a polypeptide or polynucleotide that comprises only a domain, motif, portion, or fragment of a parent or reference compound, and the polypeptide or encoded polypeptide retains at least 50% activity associated with the domain, portion, or fragment of the parent or reference compound.

In certain embodiments, a functional variant or functional portion or functional fragment each refers to a “signaling portion” of an effector molecule, effector domain, costimulatory molecule, or costimulatory domain. In other aspects, a functional variant or functional portion or functional fragment each refers to a linking function or a leader peptide function as disclosed herein. In certain aspects, a functional variant/portion/fragment refers to a linking function or a leader peptide function as described herein. In specific aspects, variant linkers and leader peptides are at least 60% as efficient, at least 70% as efficient, at least 80% as efficient, at least 90% as efficient, at least 95% as efficient, or at least 99% as efficient as the reference/parent polypeptides disclosed herein.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. Here, “plasmid,” “expression plasmid,” “virus,” and “vector” are often used interchangeably.

The terms “modify,” “modifying,” or “modification” in the context of making alterations to nucleic compositions of a cell, and the term “introduced” in the context of inserting a nucleic acid molecule into a cell, include reference to the alteration or incorporation of a nucleic acid molecule in a eukaryotic cell wherein the nucleic acid molecule may be incorporated into the genome of a cell and converted into an autonomous replicon. “Modification” or “introduction” of nucleic compositions in a cell may be accomplished by a variety of methods known in the art, including, but not limited to, transfection, transformation, transduction, or gene editing. As used herein, the term “engineered,” “recombinant,” “modified,” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications and/or introductions of expressible nucleic acid molecules encoding polypeptide, such as additions, deletions, substitutions, mutations, or other functional changes of a cell’s genetic material.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell or microorganism targeted for genetic modification with a heterologous nucleic acid molecule to produce a polypeptide of interest. In certain embodiments, a host cell may optionally already possess or be modified to include other genetic modifications that confer desired properties related, or unrelated to, biosynthesis of the heterologous protein.

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. In certain embodiments, amounts of a certain cell type in a mixture will be enriched and amounts of a different cell type will be depleted, such as enriching for CD4+ cells while depleting CD8+ cells, or enriching for CD8+ cells while depleting CD4+ cells, or combinations thereof.

“Antigen” as used herein refers to an immunogenic molecule that provokes an immune response. This immune response may involve antibody production, activation of specific immunologically-competent cells, or both. An antigen may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

The term “epitope” includes any molecule, structure, amino acid sequence, or protein determinant that is recognized and specifically bound by a cognate binding molecule, such as a chimeric antigen receptor, or other binding molecule, domain, or protein.

“Exogenous” with respect to a nucleic acid or polynucleotide indicates that the nucleic acid is part of a recombinant nucleic acid construct or is not in its natural environment. For example, an exogenous nucleic acid can be a sequence from one species introduced into another species (i.e., a heterologous nucleic acid). Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, for example, non-native regulatory sequences flanking a native sequence in a recombinant nucleic acid construct. In addition, stably transformed exogenous nucleic acids typically are integrated at positions other than the position where the native sequence is found. The exogenous elements may be added to a construct, for example, using genetic recombination. Genetic recombination is the breaking and rejoining of DNA strands to form new molecules of DNA encoding a novel set of genetic information.

A “T cell” or “T lymphocyte” is an immune system cell that matures in the thymus and produces TCRs, including αβT cells and γδT cells. T cells can be naive (not exposed to antigen; increased expression of CD62L, CCR7, CD28, CD3, CD127, and CD45RA, and decreased expression of CD45RO as compared to TCM), memory T cells (TM) (antigen-experienced and long-lived), and effector cells (antigen-experienced, cytotoxic). TM can be further divided into subsets of central memory T cells (TCM, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naive T cells or TCM).

The term “leader sequence,” used interchangeably with “signal sequence” and also referred to as “leader peptide” or “signal peptide” herein, is an amino acid sequence at the N-terminus of a peptide or a polypeptide that confers a trafficking preference to the peptide or the polypeptide, directs the nascent peptide or polypeptide to the ER, facilitates ER to Golgi transport, and/or facilitates aspects of late secretory processing. The term “leader sequence” also refers to a nucleotide sequence encoding the leader peptide.

In addition, it should be understood that the individual constructs, or groups of constructs, derived from the various combinations of the structures and subunits described herein, are disclosed by the present disclosure to the same extent as if each construct or group of constructs was set forth individually. Thus, selection of particular structures or particular subunits is within the scope of the present disclosure.

The terminology used in the description is intended to be interpreted in its broadest reasonable manner, even though it is being used in conjunction with a detailed description of identified embodiments.

### II. Second Generation Constructs and Associated Compositions

Provided herein in certain embodiments are second generation polypeptides, such as single chain trimer (SCT) polypeptides, comprising or consisting essentially of a target peptide, a first linker (e.g., L1), at least a portion of a beta-2 microglobulin domain, a second linker (e.g., L2), and at least a portion of a major histocompatibility complex (MHC) I alpha chain (e.g., MHC-alpha), or pharmaceutically acceptable derivatives thereof. In some embodiments, these SCT polypeptides are referred to as dt-SCT polypeptides or GGGAS-L1 polypeptides.

### A. Linker 1

In some aspects, the first linker of the ds-SCT polypeptide is a peptide. In some aspects, the first linker of the ds-SCT polypeptide has an amino acid sequence that is at least about 70% homologous to GCGGSGGGGSGGGGS (SEQ ID NO: 1), at least about 80% homologous to GCGGSGGGGSGGGGS (SEQ ID NO: 1), at least about 85% homologous to GCGGSGGGGSGGGGS (SEQ ID NO: 1), at least about 90% homologous to GCGGSGGGGSGGGGS (SEQ ID NO: 1), at least about 95% homologous to GCGGSGGGGSGGGGS (SEQ ID NO: 1), at least about 97.5% homologous to GCGGSGGGGSGGGGS (SEQ ID NO: 1), or at least about 99% homologous to GCGGSGGGGSGGGGS (SEQ ID NO: 1). In some aspects, the first linker ds-SCT polypeptide has an amino acid sequence that is GCGGSGGGGSGGGGS (SEQ ID NO: 1).

In some aspects, the first linker of the ds-SCT polypeptide has an amino acid sequence that is at least about 70% homologous to GCGASGGGGSGGGGSGGGGS (SEQ ID NO: 2), at least about 80% homologous to GCGASGGGGSGGGGSGGGGS (SEQ ID NO: 2), at least about 85% homologous to GCGASGGGGSGGGGSGGGGS (SEQ ID NO: 2), at least about 90% homologous to GCGASGGGGSGGGGSGGGGS (SEQ ID NO: 2), at least about 95% homologous to GCGASGGGGSGGGGSGGGGS (SEQ ID NO: 2), at least about 97.5% homologous to GCGASGGGGSGGGGSGGGGS (SEQ ID NO: 2), or at least about 99% homologous to GCGASGGGGSGGGGSGGGGS (SEQ ID NO: 2). In some aspects, the first linker of the ds-SCT polypeptide has an amino acid sequence that is GCGASGGGGSGGGGSGGGGS (SEQ ID NO: 2).

In some aspects, the first linker of the ds-SCT polypeptide has an amino acid sequence that is at least about 70% homologous to GCGASGGGGSGGGGS (SEQ ID NO: 3), at least about 80% homologous to GCGASGGGGSGGGGS (SEQ ID NO: 3), at least about 85% homologous to GCGASGGGGSGGGGS (SEQ ID NO: 3), at least about 90% homologous to GCGASGGGGSGGGGS (SEQ ID NO: 3), at least about 95% homologous to GCGASGGGGSGGGGS (SEQ ID NO: 3), at least about 97.5% homologous to GCGASGGGGSGGGGS (SEQ ID NO: 3), or at least about 99% homologous to GCGASGGGGSGGGGS (SEQ ID NO: 3). In some aspects, the first linker has an amino acid sequence that is GCGASGGGGSGGGGS (SEQ ID NO: 3). Without wishing to be bound by theory, GCGASGGGGSGGGGS (SEQ ID NO: 3) shows binding to 1G4 and variants in mammalian cells (Zhao, 2007). The first linker having an amino acid sequence that is at least about 70% or more homologous to GCGASGGGGSGGGGS (SEQ ID NO: 3) may be used to support binding to 1G4 and variants in mammalian cells.

In some aspects, at least the portion of the MHC I alpha chain of the ds-SCT polypeptide comprises an amino acid substitution compared to a wild-type MHC I alpha chain. In some aspects, the amino acid substitution is {Y84C}. In some aspects, a disulfide bridge forms between the first linker ds-SCT polypeptide and the MHC I alpha chain ds-SCT polypeptide. In some aspects, the disulfide bond forms between a cysteine reside in the first linker and a cystine residue in MHC I alpha chain. In some aspects, the disulfide bridge forms at the {G2C} of the first linker and the {Y84C} of the MHC I alpha chain.

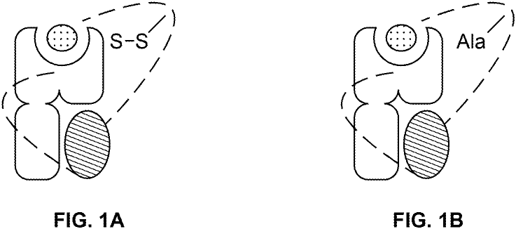

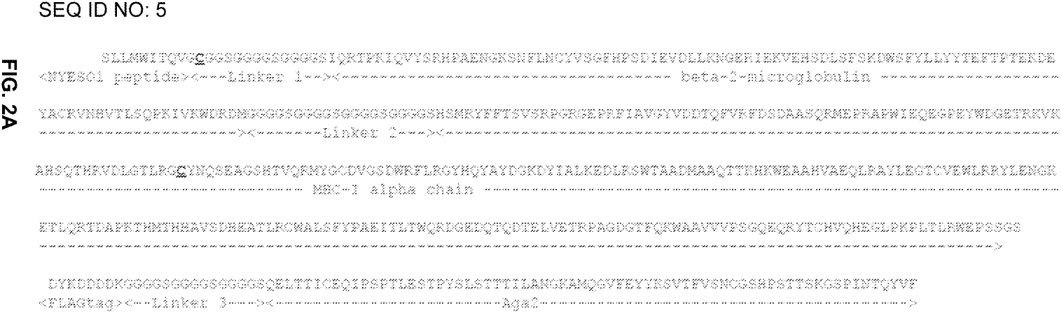

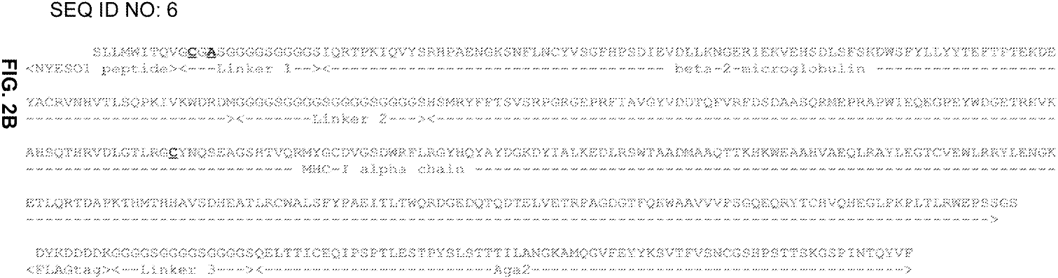

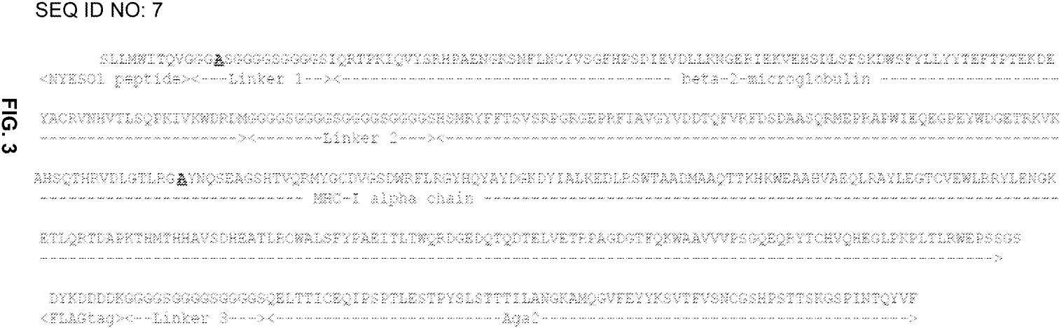

Constructs with a dt-SCT linker 1 of the present technology are conceptually illustrated in FIG. 1A. In some embodiments, the target peptide is NY-ESO-1. In these embodiments, an amino acid sequence of the dt-SCT polypeptide with the NY-ESO-1 target peptide (“NY-ESO-1 / HLA-A2 SCT”) is the amino acid sequence shown in FIG. 2A.

In some embodiments, the target peptide is NY-ESO-1. In these embodiments, an amino acid sequence of the dt-SCT polypeptide with the NY-ESO-1 target peptide (“NY-ESO-1 / HLA-A2 SCT”) is the amino acid sequence shown in FIG. 2B.

In other aspects, the first linker GGGAS-L1 polypeptides has an amino acid sequence that is at least about 70% homologous to GGGASGGGGSGGGGS (SEQ ID NO: 4), at least about 80% homologous to GGGASGGGGSGGGGS (SEQ ID NO: 4), at least about 85% homologous to GGGASGGGGSGGGGS (SEQ ID NO: 4), at least about 90% homologous to GGGASGGGGSGGGGS (SEQ ID NO: 4), at least about 95% homologous to GGGASGGGGSGGGGS (SEQ ID NO: 4), at least about 97.5% homologous to GGGASGGGGSGGGGS (SEQ ID NO: 4), or at least about 99% homologous to GGGASGGGGSGGGGS (SEQ ID NO: 4). In these aspects, the first linker GGGAS-L1 polypeptides has an amino acid sequence that is GGGASGGGGSGGGGS (SEQ ID NO: 4).

In some aspects, at least the portion of the MHC I alpha chain GGGAS-L1 polypeptides comprises an amino acid substitution compared to a wild-type MHC I alpha chain. In some aspects, the amino acid substitution is {Y84A}. In some aspects, a disulfide bridge forms between the first linker GGGAS-L1 polypeptides and the MHC I alpha chain GGGAS-L1 polypeptides. For example, the GGGAS-L1 polypeptide includes a disulfide bond between the first linker and MHC-alpha that forms between an alanine reside in the first linker and an alanine residue in MHC-alpha. In some aspects, the disulfide bond forms between the alanine residue in the first linker at {G4A} and the alanine residue in the MHC I alpha chain at {Y84A}.

Constructs with a GGGAS-Linker 1 of the present technology are conceptually illustrated in FIG. 1B. In some embodiments, the target peptide is NY-ESO-1. In these embodiments, an amino acid sequence of the GGGAS-L1 linker polypeptide with the NY-ESO-1 target peptide (“NY-ESO-1/HLA-A2 SCT”) is the amino acid sequence shown in FIG. 3.

In some embodiments, the polypeptides, such as the dt-SCT polypeptides and the GGGAS-L1 linker polypeptides comprise or consist essentially of a tag, a third linker, and/or a tether peptide. In some embodiments, the tether peptide is Aga2.

In some embodiments, the dt-SCT polypeptides and the GGGAS-L1 linker polypeptides comprise or consist essentially of a leader peptide. In some aspects, each of the dt-SCT polypeptides and the GGGAS-L1 linker polypeptides further comprise a leader sequence. In some embodiments, the leader peptide or the leader sequence directs the polypeptide to the ER, facilitates ER to Golgi transport, and/or facilitates aspects of late secretory processing. Aspects of leader peptides is further set forth elsewhere in the present disclosure.

### B. Linker 1-Free Compositions

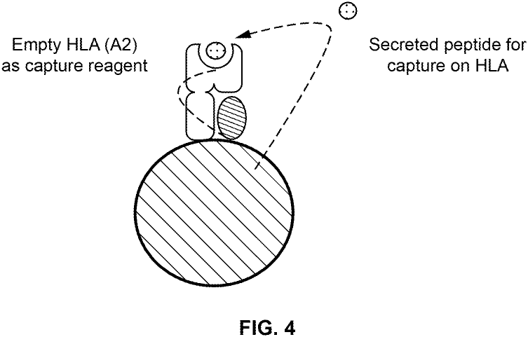

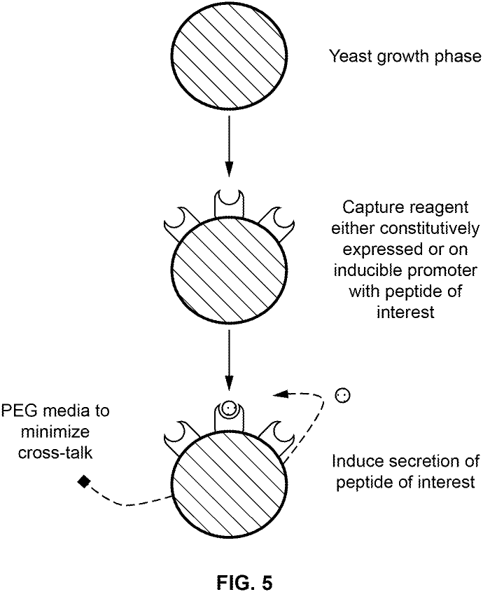

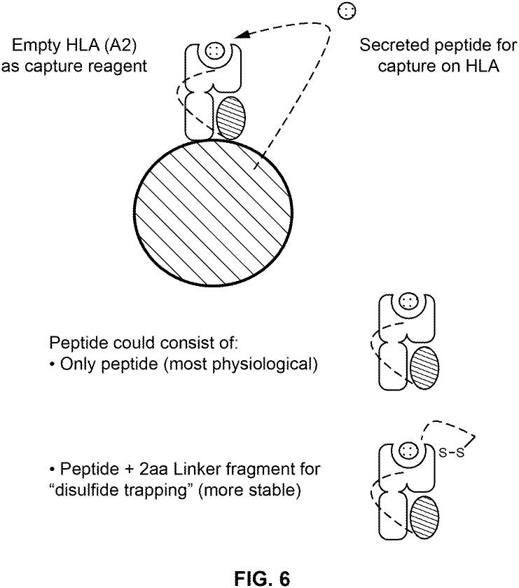

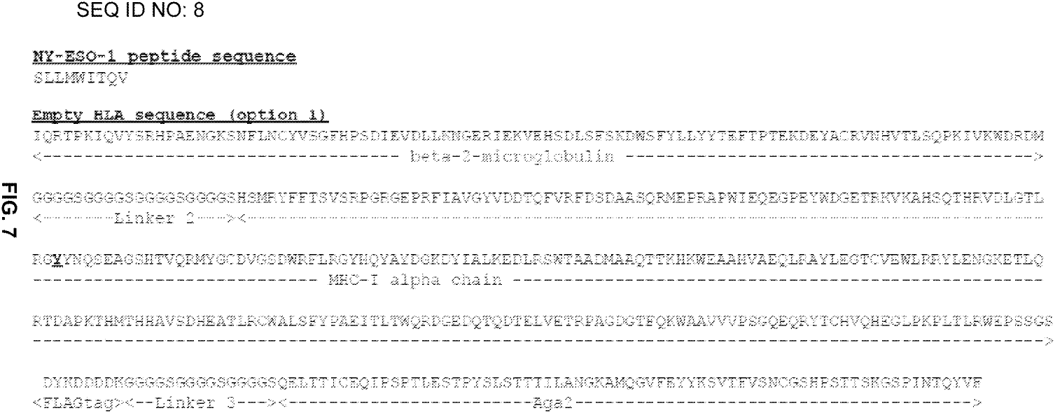

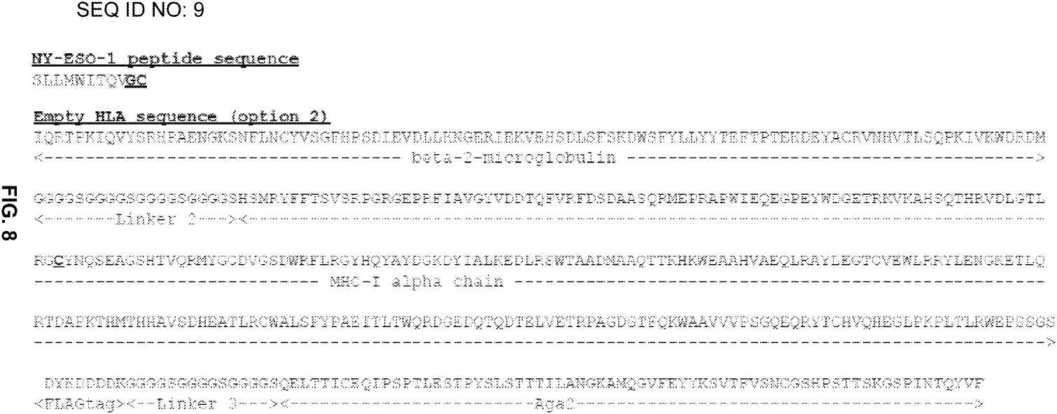

Polypeptide compositions of the present disclosure can also lack an L1 linker in any form, and rather comprise two polypeptides secreted independently of one another, and optionally expressed separately. A schematic of the linker 1-free constructs described herein is shown in FIGS. 4-6 and 11A.

In some embodiments, the polypeptide compositions comprise or consist essentially of a first polypeptide comprising the target peptide, and a second polypeptide comprising at least the portion of a beta-2 microglobulin domain, the second linker (e.g., L2), and at least the portion of a major histocompatibility complex (MHC) I alpha chain, the third linker (e.g., L3), and the tether peptide, or pharmaceutically acceptable derivatives thereof. In some embodiments, the second polypeptide has the structure B-L2-A-L3-T.

Once expressed by a cell, the first polypeptide is bound by at least a portion of the second polypeptide (“captured”). In some embodiments, the second polypeptide is expressed on a cell surface, such as a yeast cell. In these embodiments, the tether domain (e.g., Aga2) retains a least a portion of the second polypeptide within the yeast cell membrane.

### C. pMHC Modifications

In some embodiments, the MHC I alpha chain has a wild-type amino acid sequence with tyrosine at position 84. Without intending to be bound by any particular theory, it is thought that the Y84 residue mimics a physiological structure of an HLA:peptide interface. An exemplary amino acid sequence with an NY-ESO-1 target peptide is shown in FIG. 7 (SEQ ID NO: 8).

In other embodiments, the first polypeptide further comprises a peptide fragment having at least two amino acids, such as glycine and cysteine. For example, the two amino acids, G and C, are fused to a C terminus of the first polypeptide. In some embodiments, the peptide fragment increases pMHC stability and/or enhances pMHC display on a cell surface. In some embodiments, at least the portion of the MHC I alpha chain comprises an amino acid substitution compared to a wild-type MHC I alpha chain. For example, the amino acid substitution is {Y84C}. In some embodiments, a disulfide bridge forms between the peptide fragment and the MHC I alpha chain. For example, the disulfide bridge forms at between the C amino acid of the peptide fragment and the {Y84C} of the MHC I alpha chain. Without intending to be bound by any particular theory, it is thought that the disulfide bridge between the peptide fragment and the MHC I alpha chain C84 residue increases pMHC stability. An exemplary amino acid sequence with an NY-ESO-1 target peptide is shown in FIG. 8 (SEQ ID NO: 9).

Without wishing to be bound by theory, structural analysis of MHC class I alpha1 chain has shown that a {Y84A} modification in MHC alpha1 chain of SCT accommodates a linker (Mitaksov, 2007). Disulfide trapped SCT (dt-SCT) is another method to accommodate a linker, when provided with a {G2C} modification in Linker 1 and {Y84C} modification in MHC alpha1 chain. Disulfide trap may compensate for weaker F-pocket anchor in HLA-A2. In some aspects of the present disclosure, SCT polypeptides comprising MHC class I alpha1 chain with a {Y84A} modification are provided. In some aspects, dt-SCTs having a Linker 1 with {G2C} modification and MHC class I alpha1 chain with a {Y84C} modification are provided.

### D. Leader Sequences

In some aspects, SCT polypeptides of the present disclosure comprise a leader peptide. In some aspects, the leader peptide is located at the N-terminus of the target peptide.

In some aspects, polypeptide compositions of the present disclosure comprise a first polypeptide comprising a target peptide, and a second polypeptide comprising at least a portion of a beta-2 microglobulin domain, a second linker, and at least a portion of a major histocompatibility complex (MHC) I alpha chain, a third linker, and a tether peptide, or pharmaceutically acceptable derivatives thereof. In some aspects, one or both of the first polypeptide and the second polypeptide further comprise(s) a leader peptide at the N-terminus.

In some aspects, the leader peptide directs the nascent peptide or polypeptide to the ER, facilitates ER to Golgi transport, and/or facilitates aspects of late secretory processing.

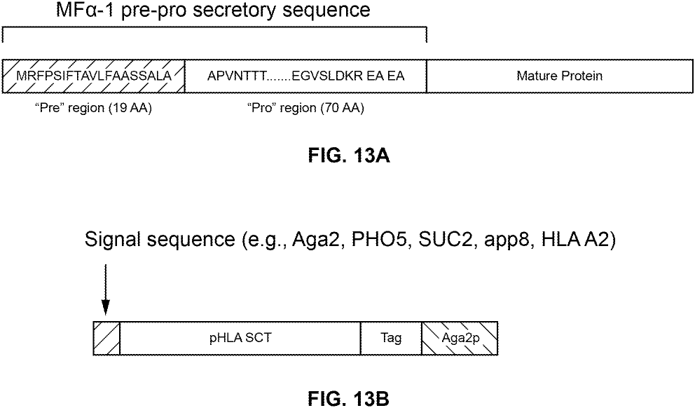

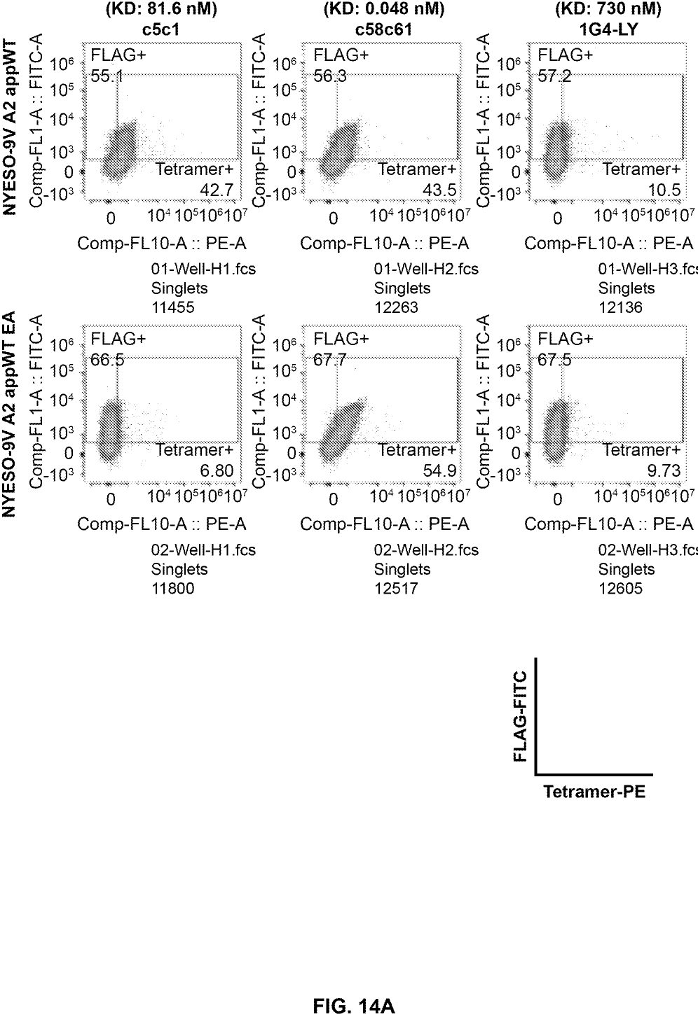

In some aspects, the leader sequence comprises a pre-pro secretory sequence. Without wishing to be bound by theory, MFα-1 (alpha mating factor 1) pre-pro secretory sequence, shown in FIG. 13A, is used for heterologous protein expression in yeast. The first 19 amino acids (“pre” region) directs the nascent polypeptide to the ER. Upon extrusion into the EF, the “pre” region is cleaved. The “pro” region facilitates ER to Golgi transport in addition to facilitate aspects of late secretory processing. Kex2p and/or Ste13p may be overwhelmed by high levels of protein expression. Secreted protein may be unprocessed or partially processed. Dipeptide spacers can improve proteolytic processing. Exemplary pre-pro sequences that may be used in the present disclosure include app8, app8EA, syn, syn EA, appWT, and appWT EA, or variants thereof. Their sequences are set forth in Table 2 below.

In another aspect, as illustrated in FIG. 13B, the leader sequence comprises an Aga2, PHO5, SUC2, app8, or HLA A2 signal sequence, or a variant thereof. PHO5 and SUC2 are yeast leader sequences that have been used for secretion of heterologous proteins. PHO5 encodes acid phosphatase. SUC2 encodes invertase. Their sequences are set forth in Table 2 below.

Exemplary leader sequences are set forth in Table 2 below [nucleotide sequences (SEQ ID NOs: 10-17); amino acid sequences (SEQ ID NOs: 18-25)].

TABLE 2

Leader Sequences

| Leader | Nucleotide Sequence | SEQ ID No: | Amino Acid Sequence | SEQ ID No: |
|---|---|---|---|---|
| PHO5 | ATGTTCAAGTCCGTCGTCTACTCTATTTTGGCTGCTTCTTTGGCTAATGCC | 10 | MFKSVVYSILAASLANA | 18 |
| SUC2 | ATGCTGCTGCAGGCCTTCTTGTTTTTGTTGGCTGGTTTTGCTGCTAAGATTTCCGCT | 11 | MLLQAFLFLLAGFAAKISA | 19 |
| app8 | ATGAGGTTCCCCTCCATTTTCACTGCTGTTTTGTTTGCTGCTTCTTCTGCTTTGGCTGCTCCAGCTAACACTACTACTGAAGATGAGACTGCTCAAATTCCAGCTGAAGCTGTTATTGATTACTCCGATTTGGAAGGTGATTTTGATGCTGCTGCTTTGCCATTGTCTAACTCTAAAACAATGGTCTGTCCTCTACCATACCACCATTGCTTCTATTGCTGCTAAAGAAGAAGGCGTTCAATTGGACAAGAGA | 12 | MRFPSIFTAVLFAASSALAAPANTTTEDETAQIPAEAVIDYSDLEGDFDAAALPLSNSTNNGLSSTNTTIASIAAKEEGVQLDKR | 20 |
| app8 EA | ATGAGGTTCCCCTCCATTTTCACTGCTGTTTTGTTTGCTGCTTCTTCTGCTTTGGCTGCTCCAGCTAACACTACTACTGAAGATGAGACTGCTCAAATTCCAGCTGAAGCTGTTATTGATTACTCCGATTTGGAAGGTGATTTTGATGCTGCTGCTTTGCCATTGTCTAACTCTACAAACAATGGTCTGTCCTCTACCAATACCACCATTGCTTCTATTGCTGCTAAAGAAGAAGGCGTTAATTGGATAAGAGAGAAGCT | 13 | MRFPSIFTAVLFAASSALAAPANTTTEDETAQIPAEAVIDYSDLEGDFDAAALPLSNSTNNGLSSTNTTIASIAAKEEGVQLDKREA | 21 |
| syn | ATGAAGGTCCTGATCGTCTTGTTGGCTATTTTTGCTGCTTTGCCATTGGCTTTGGCTCAACCAGTTATTTCTACTACTGTTGGTTCTGCTGCTGAAGGTTCTTTGGATAAGAGA | 14 | MKVLIVLLAIFAALPLALAQPVISTTVGSAAEGSLDKR | 22 |
| syn EA | ATGAAGGTCCTGATCGTCTTGTTGGCTATTTTTGCTGCTTTGCCATTGGCTTTGGCTCAACCAGTTATTTCTACTACTGTTGGTTCTGCTGCTGAAGGTTCTTTGGATAAGAGAGAAGCT | 15 | MKVLIVLLAIFAALPLALAQPVISTTVGSAAEGSLDKREA | 23 |
| appWT | ATGAGATTTCCTTCAATTTTTACTGCAGTTTTATTCGCAGCATCTCCGCATTAGCTGCTCCAGTCAACACTACAACAGAAGATGAAACGGCACAAATTCCGGCTGAAGCTGTCATCGGTTACTTAGATTTAGAAGGGGATTTCGATGTTGCTGTTTTGCCATTTTCCAACAGCACAAATAACGGGTTATTGTTTATAAATACTACTATTGCCAGCATTGCTGCTAAAGAAGAAGGGGTACAGCTGGATAAAAGA | 16 | MRFPSIFTAVLFAASSALAAPVNTTTEDETAQIPAEAVIGYLDLEGDFDVAVLPFSNSTNNGLLFINTTIASIAAKEEGVQLDKR | 24 |
| appWT EA | ATGAGATTTCCTTCAATTTTTACTGCAGTTTTATTCGCAGCATCCTCCGCATTAGCTGCTCCAGTCAACACTACAACAGAAGATGAAACGGCACAAATTCCGGCTGAAGCTGTCATCGGTTACTTAGATTTAGAAGGGGATTTCGATGTTGCTGTTTTGCCATTTTCCAACAGCACAAATAACGGGTTATTGTTTATAAATACTACTATTGCCAGCATTGCTGCTAAAGAAGAAGGGGTACAGCTGGATAAAAGAGAAGCT | 17 | MRFPSIFTAVLFAASSALA APVNTTTEDETAQIPAEAV IGYLDLEGDFDVAVLPFSN STNNGLLFINTTIASIAAKE EGVQLDKREA | 25 |

In some aspects, the leader peptide of the present disclosure shares 70% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 80% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 85% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 90% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 95% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 97.5% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide shares 99% or greater sequence identity with a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some aspects, the leader peptide comprises a sequence of any one of PHO5 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23). In some preferred aspects, the leader peptide comprises a sequence of PHO5 (SEQ ID NO: 18) or SUC2 (SEQ ID NO: 19).

In some aspects, the leader peptides of the present disclosure provide an increase in display of the SCT polypeptides to which they are attached on a cell surface compared to display of SCT polypeptides without a leader peptide or with another leader peptide, e.g., an Aga2 leader peptide. The display of SCT polypeptides may be assessed by fluorescence and tag-based detection of cell surface SCT polypeptides or functional binding assays with TCRs as described in the present disclosure, or any methods known in the art. In some aspects, the display of the SCT polypeptides provided by the leader sequences of the present disclosure is greater than 500%, about 500%, about 400%, about 300%, about 200%, about 190%, about 180%, about 170%, about 160%, about 150%, about 140%, about 130%, about 120%, or about 110% compared to display of SCT polypeptides without a leader peptide or with another leader peptide, e.g., an Aga2 leader peptide. In some embodiments, the leader peptides of the present disclosure increase display of SCT polypeptides comprising one of various target peptides, including, but not limited to, NY-ESO, AFP, MART-1, and MAGE-A4, and their binding to one or more of various TCRs, including, but not limited to, 1G4, 1G4-LY, NY7, AFP-1, AFP -2, MAGE-A4-1, MAGEA4-2, and DMF5.

### E. Contemplated SCT Polypeptides With Linker 1 Variations and Leader Sequences

Exemplary combinations of Linker 1 variations and leader sequences for SCT polypeptides in accordance with the present disclosure are set forth in Table 3 below. Combinations are not limited to those listed herein. Any other variations and combinations of a Linker 1 and a leader sequence may be included in SCT polypeptides, in addition to any variations to other components of the SCT polypeptides, in accordance with the present disclosure.

TABLE 3

Contemplated SCT Polypeptides with Linker 1 Variations and Leader Sequences

| Linker 1 | Leader Sequence |
|---|---|
| dt-SCT linker with a {G2C} substitution (e.g., SEQ ID NO: 1) | PHO5 (SEQ ID NO: 18) |
| dt-SCT linker with {G2C, G4A} substitutions (e.g., SEQ ID NOs: 2, 3) | PHO5 (SEQ ID NO: 18) |
| GGGAS linker (e.g., SEQ ID NO: 4) | PHO5 (SEQ ID NO: 18) |
| No linker | PHO5 (SEQ ID NO: 18) |
| dt-SCT linker with a {G2C} substitution (e.g., SEQ ID NO: 1) | SUC2 (SEQ ID NO: 19) |
| dt-SCT linker with {G2C, G4A} substitutions (e.g., SEQ ID NOs: 2, 3) | SUC2 (SEQ ID NO: 19) |
| GGGAS linker (e.g., SEQ ID NO: 4) | SUC2 (SEQ ID NO: 19) |
| No linker | SUC2 (SEQ ID NO: 19) |
| dt-SCT linker with a {G2C} substitution (e.g., SEQ ID NO: 1) | app8 (SEQ ID NO: 20) |
| dt-SCT linker with {G2C, G4A} substitutions (e.g., SEQ ID NOs: 2, 3) | app8 (SEQ ID NO: 20) |
| GGGAS linker (e.g., SEQ ID NO: 4) | app8 (SEQ ID NO: 20) |
| No linker | app8 (SEQ ID NO: 20) |
| dt-SCT linker with a {G2C} substitution (e.g., SEQ ID NO: 1) | app8 EA (SEQ ID NO: 21) |
| dt-SCT linker with {G2C, G4A} substitutions (e.g., SEQ ID NOs: 2, 3) | app8 EA (SEQ ID NO: 21) |
| GGGAS linker (e.g., SEQ ID NO: 4) | app8 EA (SEQ ID NO: 21) |
| No linker | app8 EA (SEQ ID NO: 21) |
| dt-SCT linker with a {G2C} substitution (e.g., SEQ ID NO: 1) | syn (SEQ ID NO: 22) |
| dt-SCT linker with {G2C, G4A} substitutions (e.g., SEQ ID NOs: 2, 3) | syn (SEQ ID NO: 22) |
| GGGAS linker (e.g., SEQ ID NO: 4) | syn (SEQ ID NO: 22) |
| No linker | syn (SEQ ID NO: 22) |
| dt-SCT linker with a {G2C} substitution (e.g., SEQ ID NO: 1) | syn EA (SEQ ID NO: 23) |
| dt-SCT linker with {G2C, G4A} substitutions (e.g., SEQ ID NOs: 2, 3) | syn EA (SEQ ID NO: 23) |
| GGGAS linker (e.g., SEQ ID NO: 4) | syn EA (SEQ ID NO: 23) |
| No linker | syn EA (SEQ ID NO: 23) |
| dt-SCT linker with a {G2C} substitution (e.g., SEQ ID NO: 1) | appWT (SEQ ID NO: 24) |
| dt-SCT linker with {G2C, G4A} substitutions (e.g., SEQ ID NOs: 2, 3) | appWT (SEQ ID NO: 24) |
| GGGAS linker (e.g., SEQ ID NO: 4) | appWT (SEQ ID NO: 24) |
| No linker | appWT (SEQ ID NO: 24) |
| dt-SCT linker with a {G2C} substitution (e.g., SEQ ID NO: 1) | appWT EA (SEQ ID NO: 25) |
| dt-SCT linker with {G2C, G4A} substitutions (e.g., SEQ ID NOs: 2, 3) | appWT EA (SEQ ID NO: 25) |
| GGGAS linker (e.g., SEQ ID NO: 4) | appWT EA (SEQ ID NO: 25) |
| No linker | appWT EA (SEQ ID NO: 25) |
| dt-SCT linker with a {G2C} substitution (e.g., SEQ ID NO: 1) | Aga2 |
| dt-SCT linker with {G2C, G4A} substitutions (e.g., SEQ ID NOs: 2, 3) | Aga2 |
| GGGAS linker (e.g., SEQ ID NO: 4) | Aga2 |
| No linker | Aga2 |
| dt-SCT linker with a {G2C} substitution (e.g., SEQ ID NO: 1) | MFα-1 pre-pro secretory sequence |
| dt-SCT linker with {G2C, G4A} substitutions (e.g., SEQ ID NOs: 2, 3) | MFα-1 pre-pro secretory sequence |
| GGGAS linker (e.g., SEQ ID NO: 4) | MFα-1 pre-pro secretory sequence |
| No linker | MFα-1 pre-pro secretory sequence |
| dt-SCT linker with a {G2C} substitution (e.g., SEQ ID NO: 1) | HLA A2 leader sequence |
| dt-SCT linker with {G2C, G4A} substitutions (e.g., SEQ ID NOs: 2, 3) | HLA A2 leader sequence |
| GGGAS linker (e.g., SEQ ID NO: 4) | HLA A2 leader sequence |
| No linker | HLA A2 leader sequence |

### III. Additional Associated Libraries, Cells, Compositions, Kits, and Methods

Also provided herein in certain aspects are libraries of polypeptides comprising or consisting essentially of at least one of the SCT polypeptides of the present disclosure and/or at least one of the polypeptide compositions of the present disclosure.

Further provided herein in certain embodiments are pharmaceutical compositions comprising or consisting essentially of at least one of the SCT polypeptides of the present disclosure and/or at least one of the polypeptide compositions of the present disclosure.

Also provided herein in certain embodiments are cells comprising or consisting essentially of at least one of the SCT polypeptides of the present disclosure and/or at least one of the polypeptide compositions of the present disclosure. In some embodiments, the cells are yeast cells, e.g., Saccharomyces cerevisiae cells.

In some embodiments, a target peptide is displayed on a cell surface by modifying the cell with the SCT polypeptides or the SCT polypeptide compositions of the present disclosure. Such modification of the cell with the SCT polypeptides or the SCT polypeptide compositions may be performed by a number of methods well known in the art, including, but not limited to, transfection, electroporation, recombination, transformation, transduction, or CRISPR gene editing.

In some embodiments, expression of the SCT polypeptides or the SCT polypeptide compositions is induced in the cells. Inducing expression of the SCT polypeptides or the SCT polypeptide compositions may be achieved by methods well known in the art, including inducing cell proliferation, expressing the SCT polypeptides or the SCT polypeptide compositions under an inducible promoter, targeting promotor sequences, or gene editing.

Further provided herein in certain embodiments are first nucleic acids comprising or consisting essentially of a second nucleic acid encoding at least one of the SCT polypeptides of the present disclosure and/or at least one of the polypeptide compositions of the present disclosure.

Also provided herein in certain embodiments are expression vectors comprising or consisting essentially of at least one of the nucleic acids of the present disclosure. In some embodiments, the nucleic acids of the present disclosure are located under an inducible promoter in the expression vector, such that the expression of the nucleic acids is inducible.

Further provided herein in certain embodiments are kits comprising or consisting essentially of a first container comprising the pharmaceutical compositions of the present disclosure in solution or in lyophilized form, optionally, a second container containing a diluent or reconstituting solution for the lyophilized formulation and instructions for (i) use of the solution or (ii) reconstitution and/or use of the lyophilized composition form.

Also provided herein in certain embodiments are methods comprising or consisting essentially of preparing one or more polypeptides selected from the group consisting of the SCT polypeptides of the present disclosure and the polypeptide compositions of the present disclosure, the method comprising co-expressing protein disulfide isomerase with one or more of the polypeptides of the present disclosure, culturing the cells of the present disclosure, and isolating the one or more polypeptides from the cell or a culture medium thereof.

In some embodiments, disulfide bond formation can be enhanced with co-expression of protein disulfide isomerase (PDI).

Further provided herein in certain embodiments are methods of displaying a target peptide on a cell surface, the method comprising modifying the cell with a first nucleic acid comprising or consisting essentially of a second nucleic acid encoding at least one of the SCT polypeptides and/or at least one of the polypeptide compositions of the present disclosure. Modifying the cell with the SCT polypeptides or the polypeptide compositions may be performed by a number of methods well known in the art, including, but not limited to, transfection, electroporation, recombination, transformation, transduction, or CRISPR gene editing. In some embodiments, the methods optionally include inducing expression of the SCT polypeptides and/or the at least one of the polypeptide compositions by, for example, inducing cell proliferation, expressing the SCT polypeptides or the SCT polypeptide compositions under an inducible promoter and activating the promotor, targeting promotor sequences, or gene editing. In some embodiments, the cells are yeast cells, e.g., Saccharomyces cerevisiae cells.

Further provided herein in certain embodiments are kits comprising or consisting essentially of a first container comprising the pharmaceutical compositions of the present disclosure in solution or in lyophilized form, optionally, a second container containing a diluent or reconstituting solution for the lyophilized formulation and instructions for (i) use of the solution or (ii) reconstitution and/or use of the lyophilized composition form.

Also provided herein in certain embodiments are in vitro methods for producing activated T cells, comprising or consisting essentially of contacting T cells with one or more of the SCT polypeptides of the present disclosure and/or one or more of the polypeptide compositions of the present disclosure.

Further provided herein in certain embodiments are activated T cells, produced by the methods of the present disclosure, that selectively recognize a cell expressing one or more peptides selected from the group consisting of the target peptides of the present disclosure.

Sequencing platforms that can be used in the present disclosure include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos BioSciences Corporation (Cambridge, Mass.) as described in U.S. application Ser. No. 11/167046, and U.S. Pat. Nos. 7,501,245; 7,491,498; 7,276,720; and in U.S. Pat. Publication Nos. US20090061439; US20080087826; US20060286566; US20060024711; US20060024678; US20080213770; and US20080103058; 3) Applied Biosystems (e.g. SOLiD sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Pat. Publication Nos. US20090029385; US20090068655; US20090024331; and US20080206764. All references are herein incorporated by reference. Such methods and apparatuses are provided here by way of example and are not intended to be limiting.

### EXAMPLES

### Example 1: dt-SCT Linker 1 Constructs

Constructs with a dt-SCT linker 1 of the present technology are conceptually illustrated in FIG. 1A. The dt-SCT design was tested using a yeast binding assay. Methods for TCR expression, yeast manipulation, and flow cytometry are described previously (Gee, 2018). Briefly, yeast display plasmids containing the dt-SCT NY-ESO-1 / HLA-A2 (FIG. 2A) construct, MART1 / HLA-A2 pMHC construct, and a variation of the dt-SCT NY-ESO-1 / HLA-A2 pMHC (FIG. 2B) construct were generated. These yeast display plasmids were transformed into EBY100, with transformants selected on the basis of the trp1 auxotrophic marker. Single colonies were grown and expression of the pMHC was induced. Biotinylated soluble versions of the NY-ESO-1 specific 1G4 TCR and the MART1-specific DMF5 TCR were used to stain induced yeast, with fluorescently labeled streptavidin as a secondary for detection by flow cytometry. DMF5 TCR has been shown to bind to clonal yeast displaying MART1/HLA-A2 pMHC (Gee, 2018), and served as a positive control.

Since the dt-SCT design requires the formation of a disulfide bond, co-expression of PDI may be beneficial for proper disulfide bond formation. PDI has been shown to improve heterologous protein expression in yeast, both for soluble secreted protein (Robinson, 1994) and yeast-displayed protein (Wang, 2018). Shuttle vectors and integrating vectors on an alternate selection marker (for example, pRS415, pRS405, respectively, both using the leu1 selection marker) will be used to express PDI under control of the constitutive promoter TEF1.

Detection of 1G4 TCR binding to clonal NY-ESO-⅟HLA-A2 yeast in the dt-SCT format may occur due to a reduction in interference of certain flexible linkers.

The dt-SCT construct will be further evaluated using additional TCR/pMHC pairs such as, but not limited to, MAGE-A4/HLA-A2, MAGE-A10/HLA-A2, PRAME/HLA-A2, AFP/HLA-A2 and MAGE-A3/HLA-A1.

### Example 2: GGGAS-Linker 1 Constructs

Constructs with a GGGAS-Linker 1 of the present technology are conceptually illustrated in FIG. 1B. The GGGAS-Linker 1 design was tested using a yeast binding assay. Methods for TCR expression, yeast manipulation, and flow cytometry are described previously (Gee, 2018). Briefly, yeast display plasmids containing GGGAS-Linker 1 NY-ESO-1 / HLA-A2 (FIG. 3) designs and MART1 / HLA-A2 pMHC were generated. These yeast display plasmids were transformed into EBY100, with transformants selected on the basis of the trp1 auxotrophic marker. Single colonies were grown and expression of the pMHC was induced. Biotinylated soluble versions of the NY-ESO-1 specific 1G4 TCR and the MART1-specific DMF5 TCR were used to stain induced yeast, with fluorescently labeled streptavidin as a secondary for detection by flow cytometry. DMF5 TCR has been shown to bind to clonal yeast displaying MART1/HLA-A2 pMHC (Gee, 2018), and served as a positive control.

Detection of 1G4 TCR binding to clonal NY-ESO-⅟HLA-A2 yeast in the GGGAS-Linker 1 format may occur due to a reduction in interference of certain flexible linkers.

The GGGAS-Linker 1 construct will be further evaluated using additional TCR/pMHC pairs such as, but not limited to, MAGE-A4/HLA-A2, MAGE-A10/HLA-A2, PRAME/HLA-A2, AFP/HLA-A2 and MAGE-A3/HLA-A1.

### Example 3: Linker 1-Free Constructs

In contrast to the dt-SCT and GGGAS-Linker 1 constructs of Examples 1 and 2, respectively, the linker 1-free construct includes co-expression of an empty HLA polypeptide (β-L2-α-L3-T) and a secreted peptide. These Linker-1 free constructs are conceptually illustrated in FIGS. 4-6 and 11A. Although the empty HLA is linked to cell surface via the T domain, the secreted peptide is not expressed as a genetic fusion protein with the HLA polypeptide.

Two options for the linker 1-free design will be evaluated. In the first option, the peptide does not have any C-terminal fusion and is the physiological peptide. In this case, the physiological peptide can be paired with MHC with tyrosine in position 84. An exemplary sequence with the NY-ESO-1 peptide for this first option is shown in FIG. 7 (SEQ ID NO: 8). In the second option, the secreted peptide can include two amino acids expressed on the C-terminus of the secreted peptide, one being G and one being C. This peptide can be paired with the MHC with a cysteine substitution at position 84 to support the formation of a disulfide bond. An exemplary sequence with the NY-ESO-1 peptide for this second option is shown in FIG. 8 (SEQ ID NO: 9).

In both options of the linker 1-free design, there is the possibility that a fraction of the secreted peptide from one yeast cell may be loaded onto the empty HLA of a neighboring cell. In the first option, there is no covalent link between the peptide and empty HLA, and as a result, dissociation from the HLA and diffusion away from the cell could cause a neighboring cell to load a peptide that it did not secrete. In the second option, inefficient disulfide bond formation may result in a similar scenario. This represents a potential break in the linkage between the genotype (plasmid encoding the DNA sequence for peptide expression) and the phenotype (peptide complexed with HLA on the yeast cell surface) and can result in limitations in a library-based selection. This “cross-talk” between cells may be overcome by addition of PEG to the induction media, which results in decreased diffusivity of the peptide. This will be evaluated and may be optimized for the linker 1 -free construct.

In order to measure cross talk, co-culture will be performed with two yeast populations, one expressing empty HLA + secreted NY-ESO-1 peptide, and one expressing empty HLA + secreted MART1 peptide. Soluble DMF5 TCR may be used to detect functional MART1/HLA-A2 complexes in a flow cytometry assay, and the level of staining on NY-ESO-1 secreting yeast could represent cross-talk.

### Example 4: Electroporation and Induction of SCT in Yeast

This example describes preparation and electroporation of yeast cells with nucleic acids encoding an exemplary SCT of the present disclosure.

### Yeast Preparation

Three or four days prior to the start of culture, yeast were streaked from glycerol stock to Yeast Peptone Dextrose (YPD) plate and grown at 30° C. On day 0, a 10 mL YPD culture was started from fresh yeast colony. On day 1, the culture was inoculated into 100 ml of pre-warmed YPD to OD=0.25, and was grown at 30° C. to OD = 1.3-1.5 for approximately 4-5 hours.

Freshly made 1 ml 2.5 M DTT in 1 M Tris pH 8, filtered through 0.2 µm pore membrane, was added to the culture, followed by addition of 10 ml 1 M LiOAc. The culture was further grown at 30° C. for 15 minutes. The culture was then centrifuged at 2500 x g for 5 min in two 50 ml conical tubes, was washed with 50 ml cold 1 M sorbitol and 1 mM CaCl2 (SoCa), and was centrifuged at 4° C. The culture was further washed with 1 ml SoCa, transferred to two 1.5 ml tubes, centrifuged at 4° C., 2000 x g for 2 min, resuspended in approximately 930 µl SoCa (into a final volume of approximately 1000 µl), and was kept on ice.

### Electroporation

For electroporation, 50 µl of yeast were mixed with ∼1 µg DNA on ice. Yeast were electroporated at 2500 V for 4-6 milliseconds with 0.5 µg of plasmids in cuvettes 2 mm, 50 µl/cuvettes. All SCT constructs used in Examples 5-9 in the present disclosure contained Y84A modification. Yeast were then washed with 1 mL YPD, cultured at 30° C. for 1 hr, resuspended in 0.5 ml SDCAA, and were plated on CM glucose minus Trp or SDCAA plates (50 ul/plate). Colonies were grown on plates at 30° C. for 2-3 days.

### Induction of SCT Expression in Yeast

Single colonies were inoculated in 500 ul SDCAA media in a 96 well deep well plate. Plates were shaken for 24 h at 450 rpm, 30° C. The next day, a frozen stock was made by adding 70 ul of the culture to 30 ul 50% glycerol, which was frozen at -80° C. in a styrofoam box.

For induction, after an average OD value is obtained from the culture, approximately 0.3 OD-ml of yeast were centrifuged in a deep well plate at 4500xg for 1 min. Supernanant was removed, and the pellet was resuspended in 300 ul SGCAA. Alternatively, if volumes are low, the yeast can be inoculated directly into SGCAA. SCT display in yeast was induced at 20° C., at 999 rpm for 24-72 hours.

### Example 5: Characterization of pHLA Expression and TCR Binding

This example describes characterizing expression of HLA peptides on yeast cells, including the yeast clones of Example 4, and functional display of antigen peptides on yeast cells. These expression measurements include FACS analysis (i) to determine the levels of peptide-MHC displayed on the surface of yeast cells; and (ii) to determine the levels of peptide-MHC binding to TCR tetramers.

### SCT Induction Levels

All SCT constructs used in the examples of the present disclosure includes HLA with a FLAG tag. At day 1 and day 2 after induction of electroporated yeast according to Example 5, the growth was checked by measuring OD600 of a few wells. Approximately 50,000 cells, or 1 ul (day 1) or 0.5 µl (day 2), of induced culture was washed with 100 ul PBS containing 0.1% BSA [PBSB (0.1%)], and was resuspended in 50 µl of anti-FLAG-FITC (1:100). Two anti-FLAG antibodies – (1) an M2 monoclonal anti-FLAG-FITC (Sigma-F4049) and (2) an anti-DYKDDDDK Tag (D6W5B)-Alexa488 (Cell Signaling 15008S) - were used. The cells and antibodies were incubated shaking at 4° C. for 1 hour. The cells were washed twice with 100 µl cold PBSB, and were resuspended in 100 µl of cold PBSB for analysis on cytometer.

### pHLA Expression and TCR Binding

On day 3 after induction, induced yeast were double stained with 500 nM TCR-tetramer, to detect functional recognition by the TCR, and 1:100 FITC-FLAG, to detect the epitope tag and display. The same protocol was also used to stain empty A2 yeast pulsed with peptide in the examples of the present disclosure.

### Yeast Stained With TCR Tetramers

TCRs were made in Expi 293 cells and biotinylated using BirA. TCRs were purified via Ni-NTA pull down, and size exclusion chromatography on an AKTA-pure using an S200 column purification.

TCR tetramer / anti-FLAG mix was prepared in PBSB (0.1%). To prepare 500 nM TCR-PE, Streptavidin-phycoerythrin (PE streptavidin; SA-PE) (BioLegend cat no. 405245) were mixed with TCR tetramers at a 1:5 ratio, i.e., 500 nM SA-PE with 2500 nM TCR. For 1G4-LY, the tetramer was mixed with SA-PE at a 1:3.5 ratio, i.e., 500 nM SA-PE with 1765 nM TCR. The following TCRs were tested: 1G4-LY, c5c1, c58c61, AFP-1, AFP-2, MAGE-A4, 1G4 WT, UQK, and NY7. An anti-FLAG-M2-FITC antibody (Sigma cat no. F4049) was then added to at a final concentration of 1:100. The mixture was incubated for 15 minutes.

Yeast growth was checked by measuring OD600 of a few wells. Approximately 50,000 cells or 0.5 µl of induced culture was washed with 100 µl PBSB (0.1%), centrifuged at 3000 x g, and 50 µl of TCR/anti-FLAG mixture prepared in (1) or (2) was added. The cells were incubated shaking at 4° C. for 1 hour. The cells were washed twice with 100 µl cold PBE, and were resuspended in 100 µl of cold PBSB for analysis on cytometer.

As a positive tetramer binding control, NY-ESO peptide was added to the empty wells. Six (6) ul of 10 mM NY-ESO peptide was mixed with 18 µl buffer. Two (2) µl of the peptide was added to cells to produce 100 µl and a final concentration of 50 µM. The mixture was incubated at 4° C. for 30 minutes, and was stained according to the protocol disclosed above.

### Example 6: Effect of Linker 1 on TCR Binding to SCTs

This example describes the effect of Linker 1 on binding of TCRs to SCTs.

### Experimental Methods

Empty A2 yeast were pulsed with FLAG-FITC-tagged SCTs (25 µM) for 2 hours at 4° C. Cells were then stained with TCR-phycoerythrin (TCR-PE) to detect functional recognition by the TCR, and with FITC-conjugated anti-FLAG antibody to detect the epitope tag and display, and were analyzed by flow cytometry as described above.

### Results

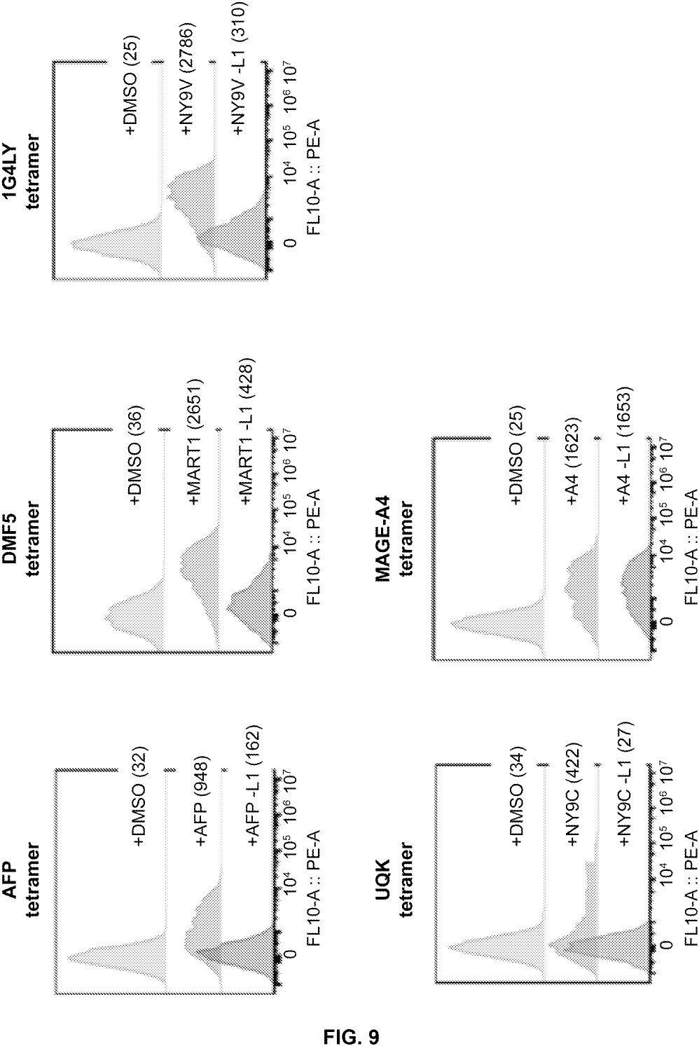

As shown in FIG. 9, all TCR tetramers (AFP, DMF5, 1G4LY, UQK) except MAGE-A4 were sensitive to Linker 1, losing binding to their peptide antigen in the absence of Linker 1. MAGE-A4 showed binding to the MAGE-A4 peptide with or without Linker 1.

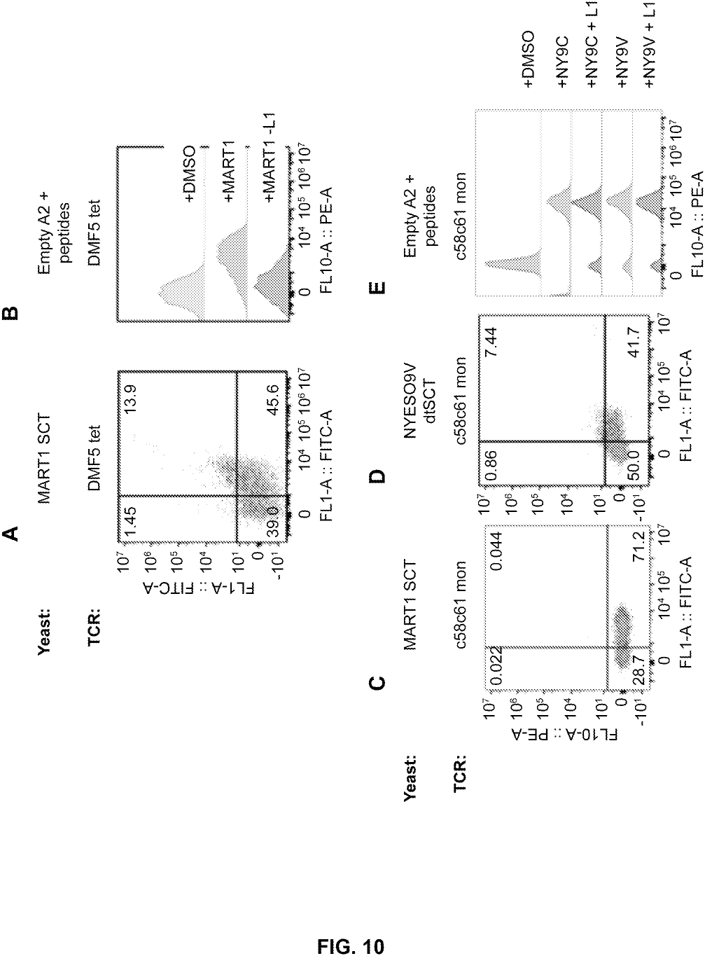

However, TCR sensitivity to Linker 1 did not always predict clonal yeast binding. As shown in FIG. 10B, the DMF5 tetramer was sensitive to Linker 1 (i.e., in the absence to Linker 1, it did not bind to the MART1 peptide pulsed to empty A2 yeast). In contrast, as shown in FIG. 10A, the DMF5 tetramer bound to clonal MART1 yeast. As shown in FIG. 10E, the c58c61 monomer (high affinity 1G4 variant with Kd of approximately 50 pM) was insensitive to Linker 1 (i.e., it bound to NY-ESO-9C and NY-ESO-9V peptides with or without Linker 1). In contrast, as shown in FIG. 10D, the c58c61 monomer did not bind clonal NY-ESO-9V dt-SCT yeast. FIG. 10C shows no binding control.

### Example 7: Recovery of Functional Display and Recognition by Using Pulsed Peptide on Yeast

This example describes the loss of TCR binding to clonal yeast SCT/Y84A, and recovery of functional display and recognition in empty A2 yeast pulsed with peptide.

### Methods

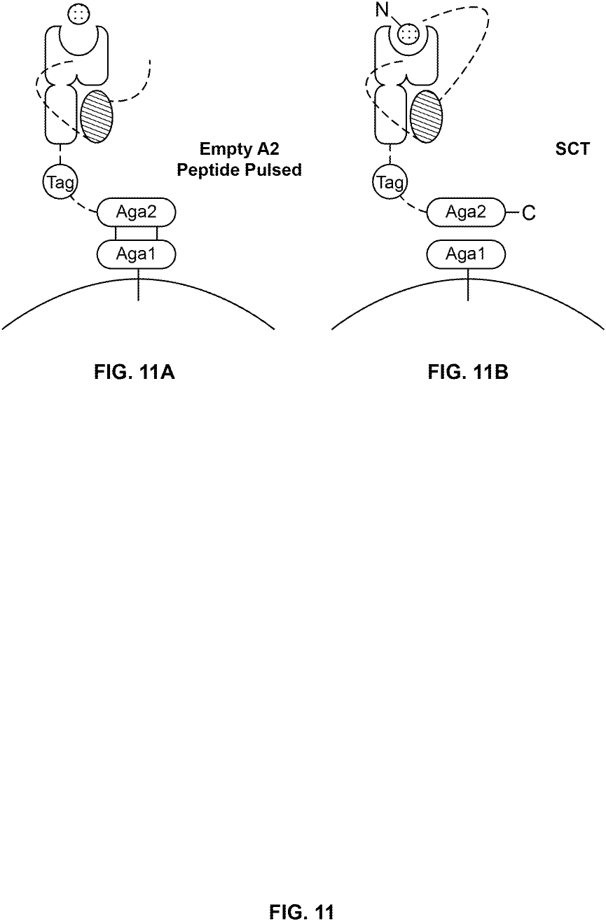

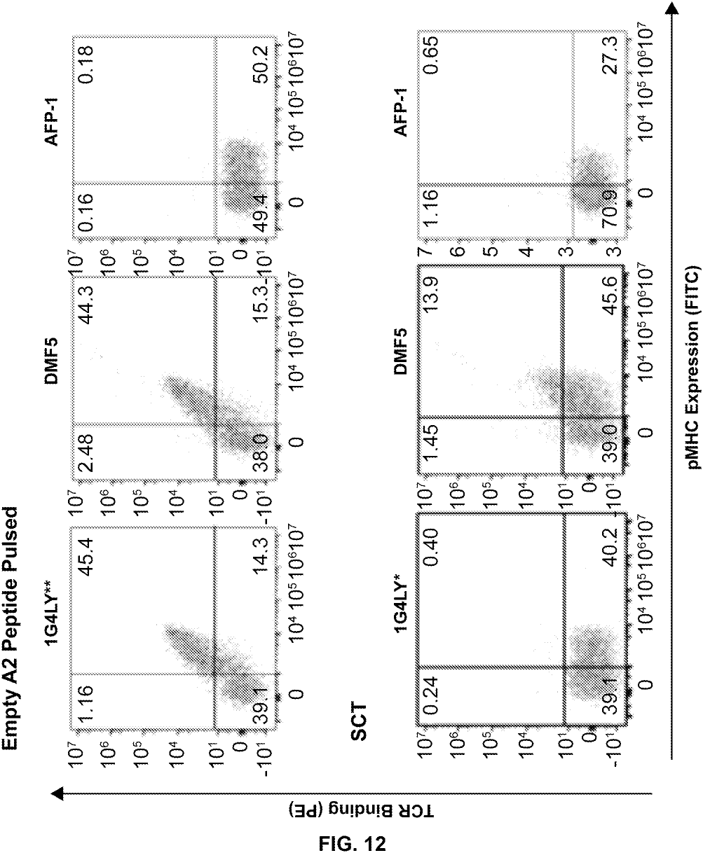

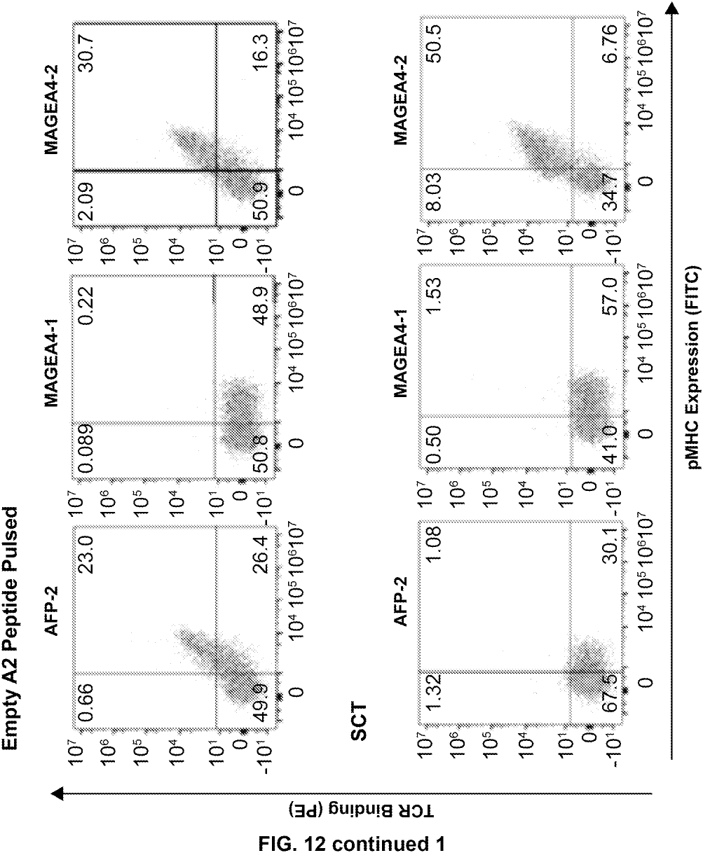

Empty A2 yeast were pulsed with 25 µM peptides for 2 hours at 4° C. as illustrated in FIG. 11A. Pulsed peptides were, from left to right in FIG. 12 top row panels, NY-ESO, MART-1, AFP, AFP, MAGE-A4, and MAGE-A4. The cells were then stained with 400 nM TCR tetramer (400 nM PE-streptavidin and 2.5 µM TCR) and an FITC-conjugated anti-FLAG antibody, and were analyzed by flow cytometry as described above.

In parallel, clonal yeast expressing SCT/Y84A as illustrated in FIG. 11B was stained with 400 nM TCR tetramer (400 nM PE-streptavidin and 2.5 µM TCR) and an anti-FLAG-FITC antibody, and were analyzed by flow cytometry as described above. Peptides contained in SCTs were, from left to right in FIG. 12 bottom row panels, NY-ESO-9V, MART-1, AFP, AFP, MAGE-A4, and MAGE-A4.

### Results

As shown in FIG. 12 bottom row panels, SCT/Y84A expressed on clonal yeast lost binding to the 1G4LY, DMF5, and AFP-2 TCRs. The binding to each TCR was recovered in empty A2 yeast pulsed with the respective peptides. The AFP-1 and MAGE-A4 1a1b TCRs showed no binding to clonal yeast transformed with SCT/Y84A, and use of pulsed peptides on empty A2 yeast did not recover binding. The MAGE-A4 4a2b TCR showed similar binding to both pulsed peptides and clonal SCTs.

### Example 8: Effect of Leader Sequences on TCR Binding to NY-ESO Peptide

This example describes the effect of leader sequences, which are alternatives to Aga2 leader sequences, on SCT display and recognition, focusing on NY-ESO SCTs.

### Experimental Methods

Yeast clones containing the NY-ESO-9V-A2-FLAG construct with the following pre-pro secretory sequences at the N-terminus of the SCT were generated and tested: appWT, appWT EA, app8, app8EA, syn, and synEA. The appWT pre-pro secretory sequence is illustrated in FIG. 13A. Further, as illustrated in FIG. 13B, yeast clones containing the NY-ESO-9V-A2-FLAG construct with the following leader sequence 5” to the SCT were generated and tested: Aga2, PHO5, SUC2, app8, app8 EA, syn, syn EA, appWT, and appWT EA. An NY-ESO-9V-A2-FLAG construct with GGGAS linker was also tested. Nucleotide and amino acid sequences of the tested leader sequences are set forth in Table 2 [nucleotide sequences (SEQ ID NOs: 10-17); amino acid sequences (SEQ ID NOs: 18-25)].

Yeast clones were induced to display SCTs, and were subsequently stained with TCR-phycoerythrin (TCR-PE) to detect functional recognition by the TCR, and with FITC-conjugated anti-FLAG antibody to detect the epitope tag and display, and were analyzed by flow cytometry as described above.

### Results

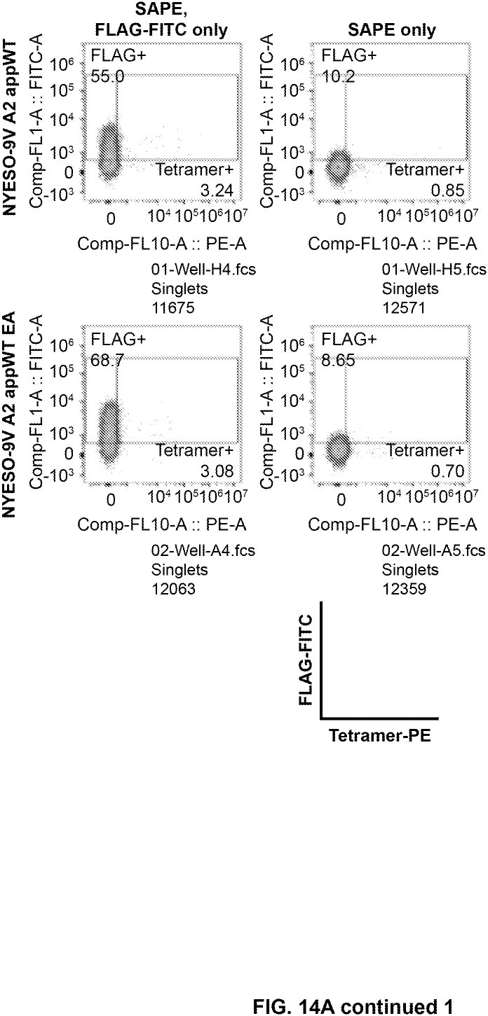

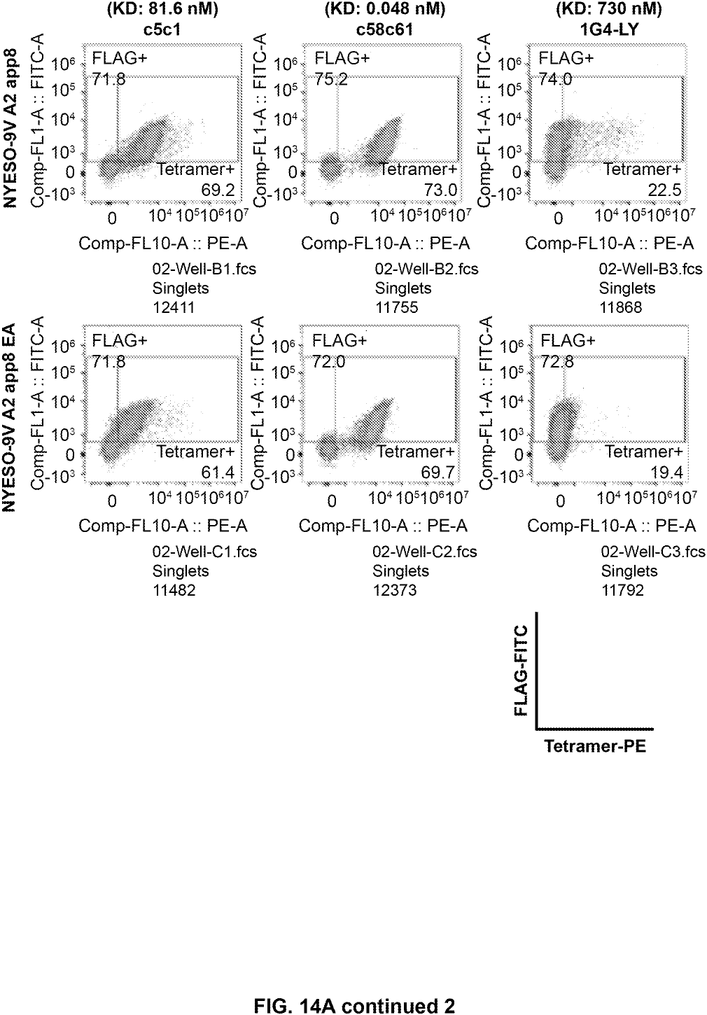

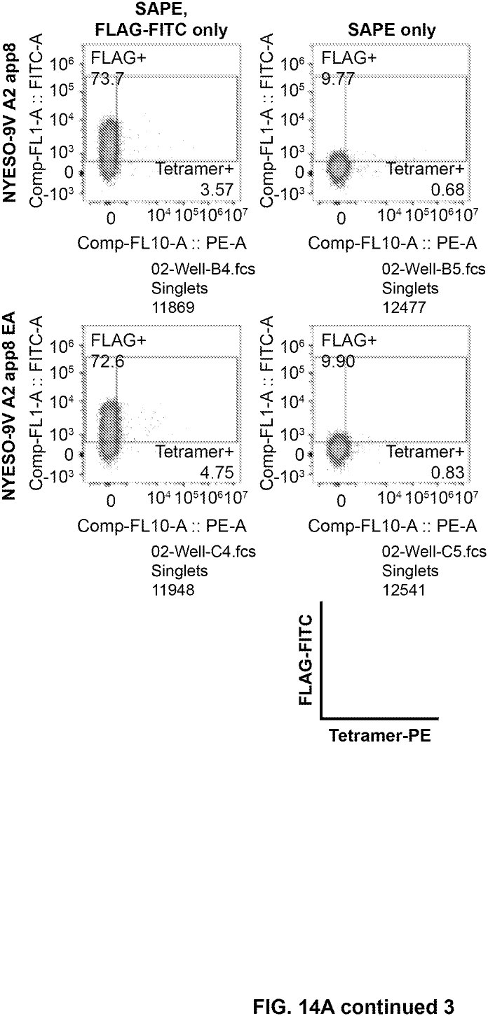

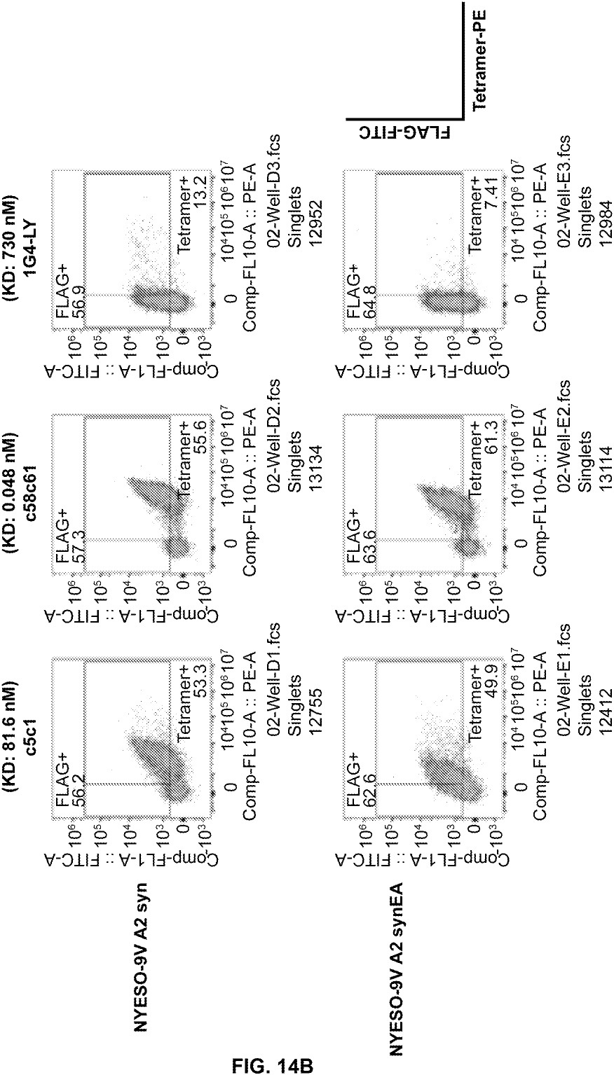

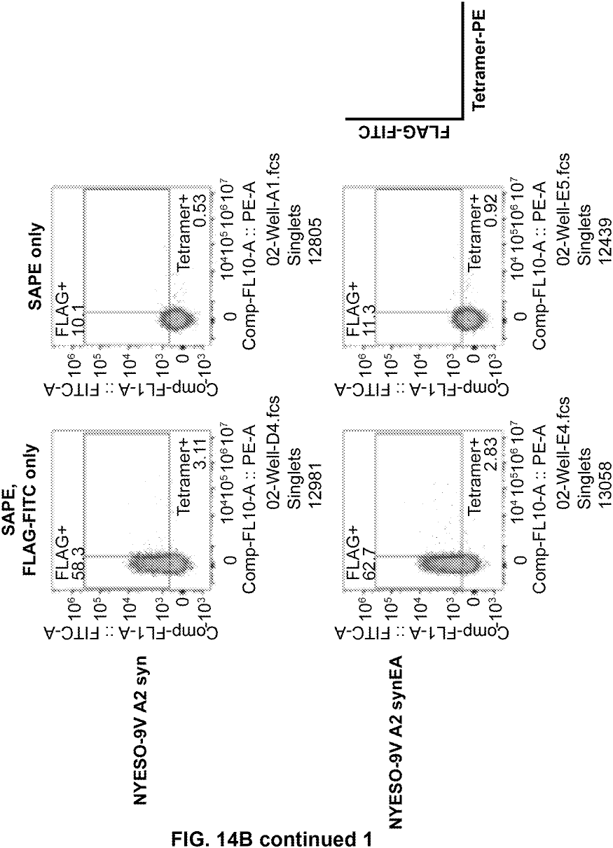

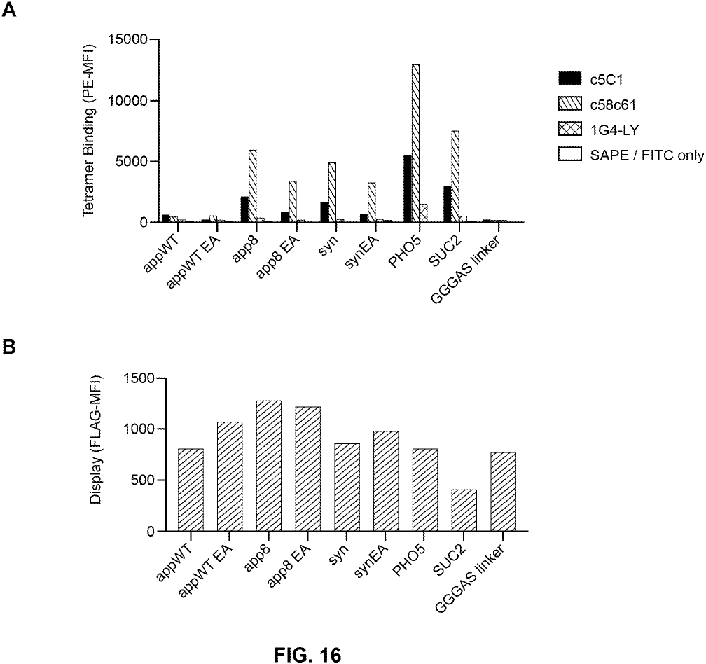

As shown in FIGS. 14A and 14B, and quantitated in FIG. 16, insertion of the pre-pro secretory sequences app8, app8 EA, syn, and syn EA rescued binding of TCRs (c5c1 and c58c61) to the NY-ESO SCTs. The app WT and appWT EA sequences also showed a small improved binding to TCR c58c61.

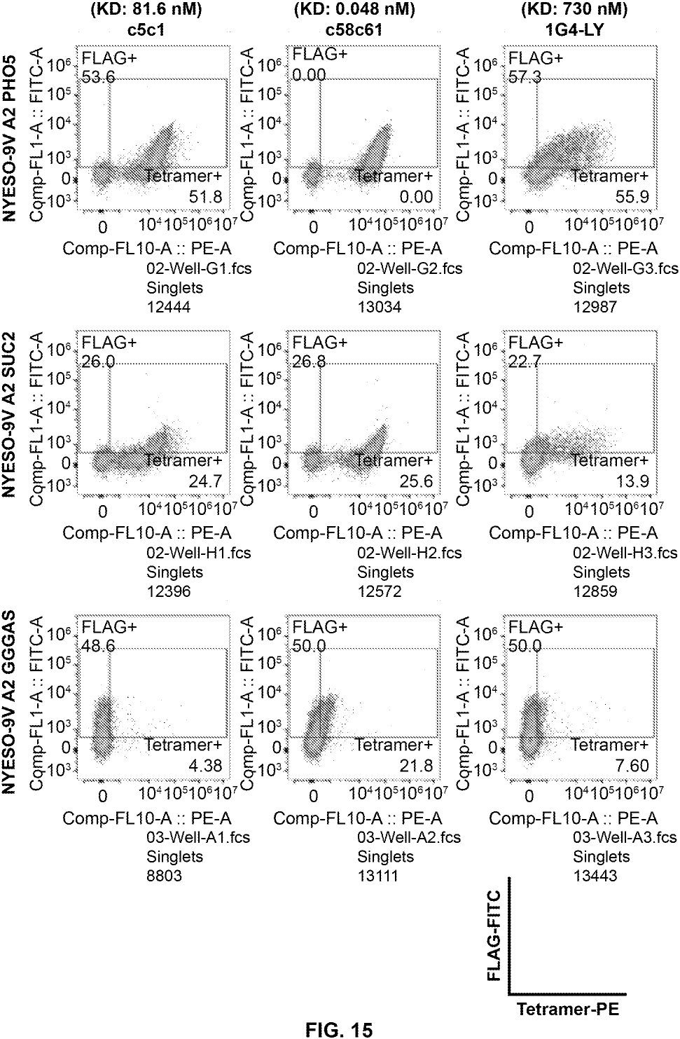

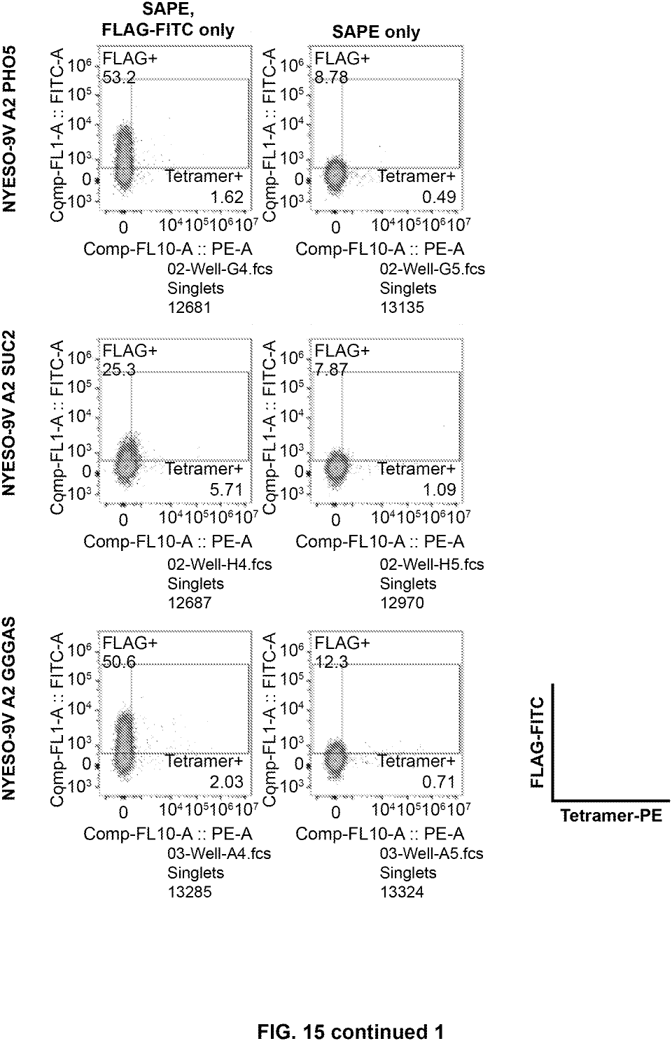

As shown in FIG. 15, insertion of PHO5 and SUC2 secretory sequences rescued binding of TCRs (c5c1, c58c61, 1G4-LY) to the NY-ESO SCTs. The GGGAS linker did not rescue the binding.

As shown in FIG. 16, PHO5 secretory sequence displayed the most robust rescue of NY-ESO SCT binding to the TCRs (c5c1, c58c61, 1G4-LY).

### Example 9: Effect of PHO5 and SUC2 Leader Sequences on TCR Binding

This example describes effect of PHO5 and SUC2 leader sequences on display and recognition of a variety of SCTs.

### Experimental Methods

Yeast clones containing the following SCT/Y84As were tested: PHOS-NY-ESO (having a PHO5 leader sequence and a NY-ESO peptide), SUC2-NY-ESO (having a SUC2 leader sequence and a NY-ESO peptide), PHO5-MART-1 (having a PHO5 leader sequence and a MART-1 peptide), SUC2-MART-1 (having a SUC2 leader sequence and a MART-1 peptide), PHO5-MART-1-cyclic (having a PHO5 leader sequence and a MART-1-cyclic peptide), SUC2-MART-1-cyclic (having a SUC2 leader sequence and a MART-1-cyclic peptide), PHO5-AFP (having a PHO5 leader sequence and a AFP peptide), SUC2-AFP (having a SUC2 leader sequence and a AFP peptide), PHOS-MAGE-A4 (having a PHO5 leader sequence and a MAGE-A4 peptide), and SUC2-MAGE-A4 (having a SUC2 leader sequence and a MAGE-A4 peptide).

Yeast clones were induced to display SCTs, and were subsequently stained with TCR-phycoerythrin (TCR-PE) to detect functional recognition by the TCR, and with FITC-conjugated anti-FLAG antibody to detect the epitope tag and display, and were analyzed by flow cytometry as described above.

### Results

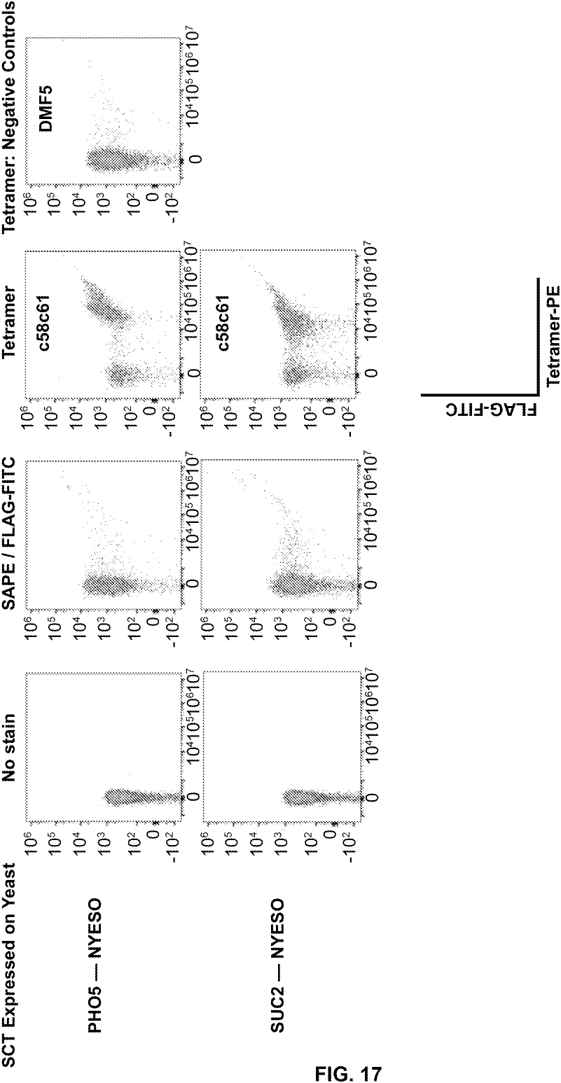

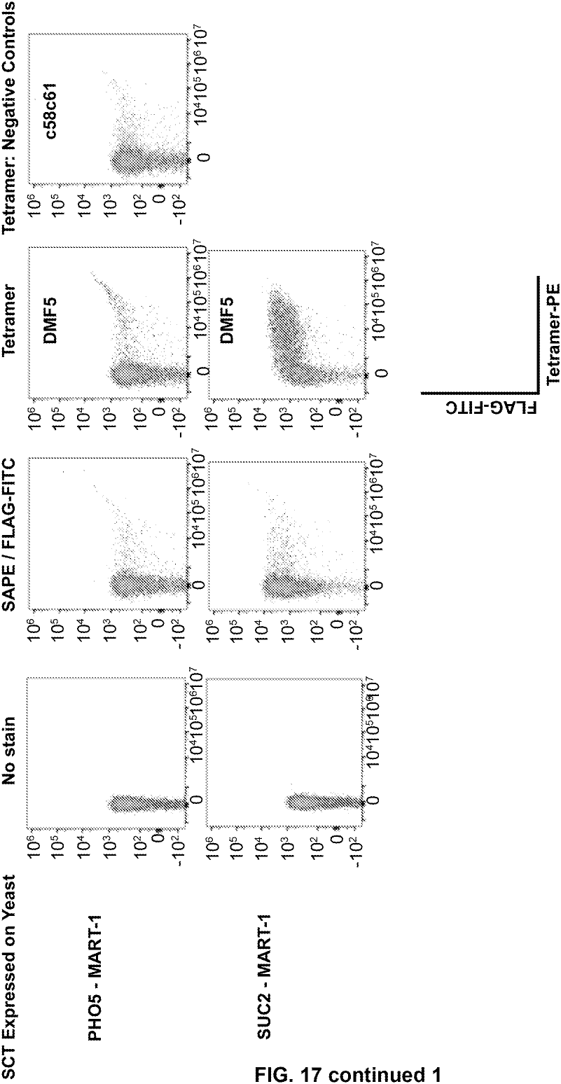

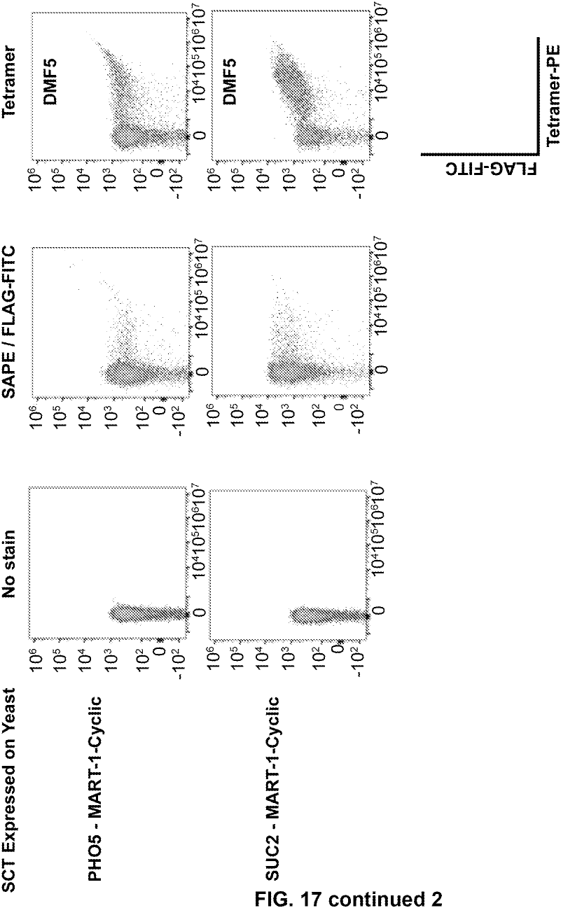

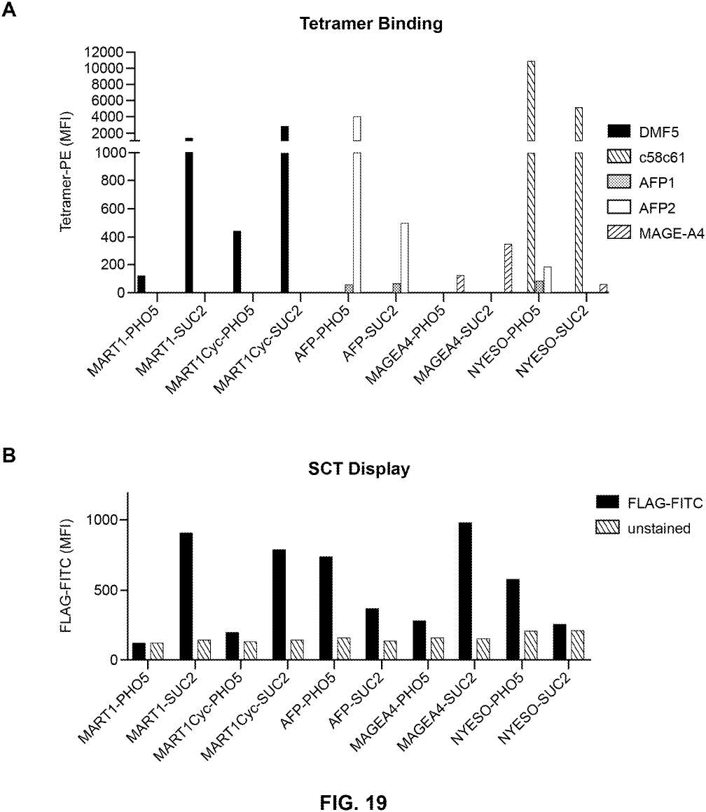

As shown in FIGS. 17-19, PHO5 and SUC2 leader sequences produced binding of NY-ESO SCT to c58c61 TCR compared to DMF5 TCR (negative control). This is consistent with data of the present disclosure in FIGS. 15-16 and Example 8. As shown in FIGS. 17 and 19, PHO5 and SUC2 leader sequences produced binding of MART-1 and MART-1-cyclic SCT to DMF5 TCR compared to c58c61 TCR (negative control).

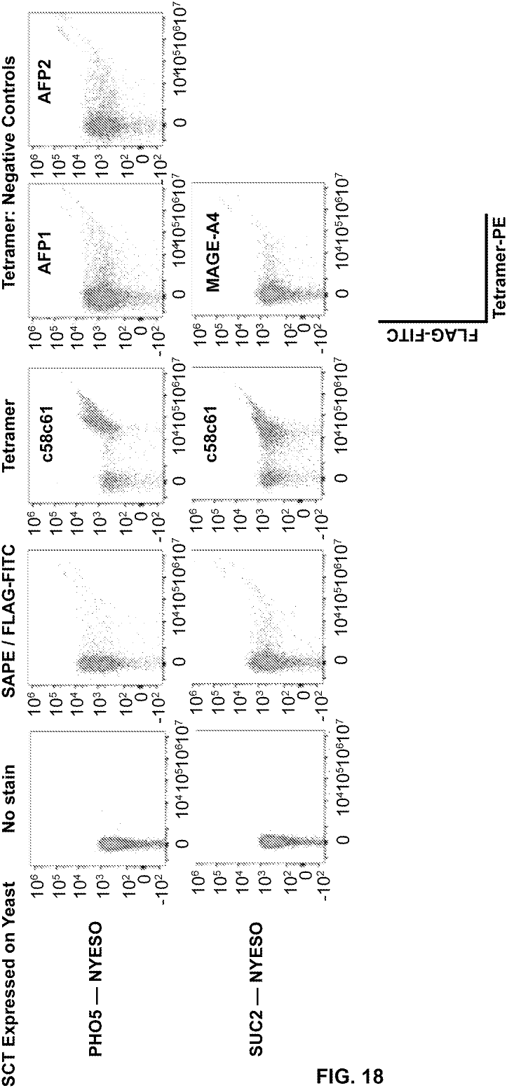

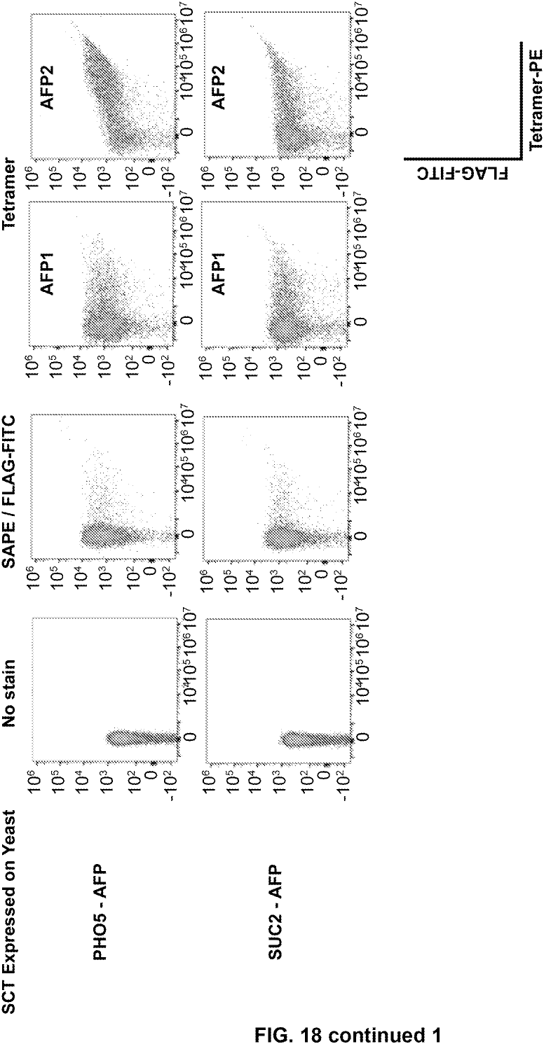

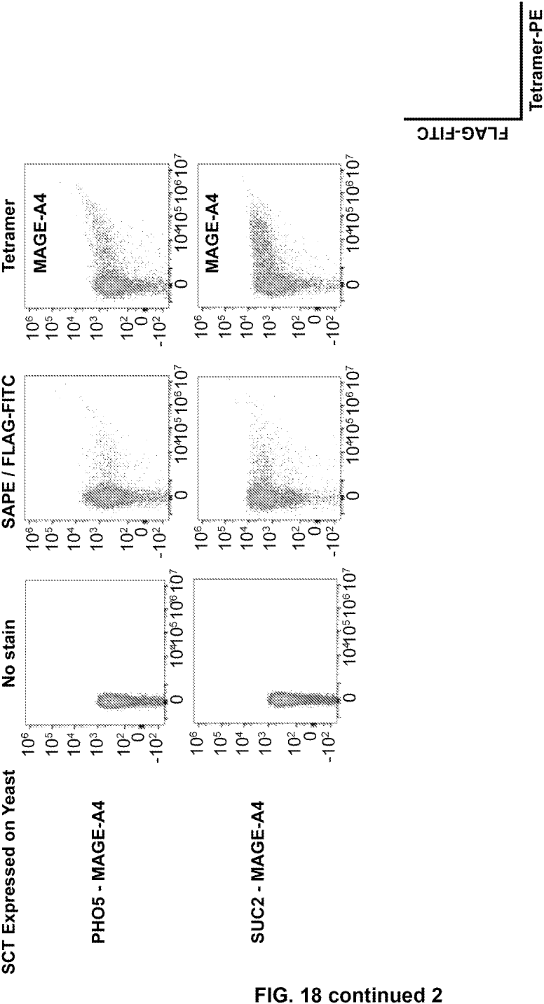

As shown in FIGS. 18 and 19, PHO5 and SUC2 leader sequences produced binding of AFP SCT to AFP-1 and AFP-2 TCR, and binding of MAGE-A4 SCT to compared to MAGE-A4.

As shown in FIG. 19, introduction of a PHO5 leader sequence produced more robust SCT display as well as SCT binding to its specific target TCR in AFP SCT (to AFP-2 TCR) and in NY-ESO SCT (to c58c61 TCR) than a SUC2 leader sequence. Introduction of a SUC2 leader sequence produced more robust SCT display as well as SCT binding to its specific target TCR in MART-1 SCT and in MART-1-cyclic SCT (to DMF5 TCR).

The results show that rescue of SCT binding to TCRs by insertion of the PHO5 or SUC2 leader sequence is not specific to the NY-ESO peptides, but it applies to all TCRs tested. Yeast display libraries with PHO5 and SUC2 signal sequences will be further developed and evaluated using additional TCRs such as, but not limited to, 1G4, 1G4-LY, NY7, AFP-1, AFP -2, MAGE-A4-1, MAGEA4-2, and DMF5, and using peptides such as, but not limited to, NY-ESO, AFP, MART-1, and MAGE-A4.

As demonstrated by the above examples, insertion of the PHO5, SUC2, app8, app8 EA, syn, or syn EA leader sequences in the context of peptide-HLA display resulted in more robust TCR binding to the peptide HLA compared to the previous Aga2 leader sequence, promoting the functional display and/or recognition by a TCR of [peptide]-[beta-2-microglobulin]-HLA.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### REFERENCES

Yu Y et al. J Immunol. 168: 3145-9 (2002).
Lybarger L et al. J Biol Chem. 278: 27104-11 (2003).
Adams JJ et al. Immunity. 35: 681-93 (2011).
Birnbaum ME et al. Cell. 157: 1073-87 (2014).
Gee M et al. Cell. 172: 549-63 (2018).
Robinson J et al. Nucleic Acids Research 39 Suppl 1:D1171-6 (2011).
Robinson AS, et al. Biotechnol. 12: 381-4 (1994).
Wang B et al. Nat Biotechnol. 36:152-5 (2018).
Rakestraw JA et al. Biotechnol Prog. 22: 1200-8 (2006).
Mitaksov V et al. Chem Biol. 14 (8): 909-22 (2007).
Zhao Y et al. J Immunol. 179(9) 5845-5854 (2007).

1. A single chain trimer (SCT) polypeptide comprising or consisting essentially of a target peptide, a first linker, at least a portion of a beta-2 microglobulin domain, a second linker, and at least a portion of a major histocompatibility complex (MHC) I alpha chain; or a pharmaceutically acceptable derivative thereof.

2. The SCT polypeptide of claim 1, wherein the first linker is a peptide.

3. The SCT polypeptide of claim 1, wherein the first linker has an amino acid sequence that is at least about 70% homologous to GCGGSGGGGSGGGGS (SEQ ID NO: 1), at least about 80% homologous to GCGGSGGGGSGGGGS (SEQ ID NO: 1), at least about 85% homologous to GCGGSGGGGSGGGGS (SEQ ID NO: 1), at least about 90% homologous to GCGGSGGGGSGGGGS (SEQ ID NO: 1), at least about 95% homologous to GCGGSGGGGSGGGGS (SEQ ID NO: 1), at least about 97.5% homologous to GCGGSGGGGSGGGGS (SEQ ID NO: 1), or at least about 99% homologous to GCGGSGGGGSGGGGS (SEQ ID NO: 1).

4. The SCT polypeptide of claim 3, wherein the first linker has an amino acid sequence that is GCGGSGGGGSGGGGS (SEQ ID NO: 1).

5. The SCT polypeptide of claim 1, wherein the first linker has an amino acid sequence that is at least about 70% homologous to GCGASGGGGSGGGGSGGGGS (SEQ ID NO: 2), at least about 80% homologous to GCGASGGGGSGGGGSGGGGS (SEQ ID NO: 2), at least about 85% homologous to GCGASGGGGSGGGGSGGGGS (SEQ ID NO: 2), at least about 90% homologous to GCGASGGGGSGGGGSGGGGS (SEQ ID NO: 2), at least about 95% homologous to GCGASGGGGSGGGGSGGGGS (SEQ ID NO: 2), at least about 97.5% homologous to GCGASGGGGSGGGGSGGGGS (SEQ ID NO: 2), or at least about 99% homologous to GCGASGGGGSGGGGSGGGGS (SEQ ID NO: 2).

6. The SCT polypeptide of claim 1, wherein the first linker has an amino acid sequence that is GCGASGGGGSGGGGSGGGGS (SEQ ID NO: 2).

7. The SCT polypeptide of claim 1, wherein the first linker has an amino acid sequence that is at least about 70% homologous to GCGASGGGGSGGGGS (SEQ ID NO: 3), at least about 80% homologous to GCGASGGGGSGGGGS (SEQ ID NO: 3), at least about 85% homologous to GCGASGGGGSGGGGS (SEQ ID NO: 3), at least about 90% homologous to GCGASGGGGSGGGGS (SEQ ID NO: 3), at least about 95% homologous to GCGASGGGGSGGGGS (SEQ ID NO: 3), at least about 97.5% homologous to GCGASGGGGSGGGGS (SEQ ID NO: 3), or at least about 99% homologous to GCGASGGGGSGGGGS (SEQ ID NO: 3).

8. The SCT polypeptide of claim 1, wherein the first linker has an amino acid sequence that is GCGASGGGGSGGGGS (SEQ ID NO: 3).

9. The SCT polypeptide of claim 1, wherein the at least a portion of the MHC I alpha chain comprises an amino acid substitution compared to a wild-type MHC I alpha chain.

10. The SCT polypeptide of claim 9, wherein the amino acid substitution is {Y84C}.

11. The SCT polypeptide of claim 9, wherein the amino acid substitution is {Y84A}.

12. The SCT polypeptide of claim 1, wherein the second amino acid counted from the N-terminus of the first linker is C.

13. The SCT polypeptide of claim 1, wherein the first linker has an amino acid substitution {G2C}.

14. The SCT polypeptide of claim 1, wherein a disulfide bridge forms between the first linker and the MHC I alpha chain.

15. The SCT polypeptide of claim 14, wherein the disulfide bridge forms at (i) the {G2C} of the first linker, or the second amino acid counted from the N-terminus of the first linker, wherein the second amino acid is C; and (ii) the {Y84C} of the MHC I alpha chain.

16. The SCT polypeptide of claim 1, wherein the first linker has an amino acid sequence that is at least about 70% homologous to GGGASGGGGSGGGGS (SEQ ID NO: 4), at least about 80% homologous to GGGASGGGGSGGGGS (SEQ ID NO: 4), at least about 85% homologous to GGGASGGGGSGGGGS (SEQ ID NO: 4), at least about 90% homologous to GGGASGGGGSGGGGS (SEQ ID NO: 4), at least about 95% homologous to GGGASGGGGSGGGGS (SEQ ID NO: 4), at least about 97.5% homologous to GGGASGGGGSGGGGS (SEQ ID NO: 4), or at least about 99% homologous to GGGASGGGGSGGGGS (SEQ ID NO: 4).

17. The SCT polypeptide of claim 16, wherein the first linker has an amino acid sequence that is GGGASGGGGSGGGGS (SEQ ID NO: 4).

18. The SCT polypeptide of claim 1, further comprising a tag, a third linker, and/or a tether peptide.

19. The SCT polypeptide of claim 1, further comprising a leader peptide.

20. The SCT polypeptide of claim 19, wherein the leader peptide directs the SCT polypeptide to the ER, facilitates ER to Golgi transport, and/or facilitates aspects of late secretory processing.

21. The SCT polypeptide of claim 19, wherein the leader peptide shares 70% or greater sequence identity with a sequence of any one of PH05 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23);
80% or greater sequence identity with a sequence of any one of PH05 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23);
85% or greater sequence identity with a sequence of any one of PH05 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23);
90% or greater sequence identity with a sequence of any one of PH05 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23);
95% or greater sequence identity with a sequence of any one of PH05 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23);
97.5% or greater sequence identity with a sequence of any one of PH05 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23); or 99% or greater sequence identity with a sequence of any one of PH05 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23).

22. The SCT polypeptide of claim 19, wherein the leader peptide comprises a sequence of any one of PH05 (SEQ ID NO: 18), SUC2 (SEQ ID NO: 19), app8 (SEQ ID NO: 20), app8 EA (SEQ ID NO: 21), syn (SEQ ID NO: 22), and syn EA (SEQ ID NO: 23).

23. The SCT polypeptide of claim 19, wherein the leader peptide comprises a sequence of PH05 (SEQ ID NO: 18) or SUC2 (SEQ ID NO: 19).

24. The SCT polypeptide of claim 18, wherein the tether peptide is Aga2.

25-51. (canceled)


SEQUENCE LISTING (25 sequences)

SEQ ID NO: 1 - protein, 15 aa, Artificial Sequence
  Linker 1
GCGGSGGGGSGGGGS

SEQ ID NO: 2 - protein, 20 aa, Artificial Sequence
  Linker 1
GCGASGGGGSGGGGSGGGGS

SEQ ID NO: 3 - protein, 15 aa, Artificial Sequence
  Linker 1
GCGASGGGGSGGGGS

SEQ ID NO: 4 - protein, 15 aa, Artificial Sequence
  Linker 1
GGGASGGGGSGGGGS

SEQ ID NO: 5 - protein, 513 aa, Artificial Sequence
  NY-ESO-1 single chain trimer having a disulfide trapped linker at a Linker 1 position with a {G2C} substitution and a {Y84C} substitution
SLLMWITQVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSGSDYKDDDDKGGGGSGGGGSGGGGSQELTTICEQIPSPTLESTPYSLSTTTILANGKAMQGVFEYYKSVTFVSNCGSHPSTTSKGSPINTQYVF

SEQ ID NO: 6 - protein, 513 aa, Artificial Sequence
  NY-ESO-1 single chain trimer having a disulfide trapped linker at a Linker 1 position with {G2C}, {G4A} substitutions and a {Y84C} substitution
SLLMWITQVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSGSDYKDDDDKGGGGSGGGGSGGGGSQELTTICEQIPSPTLESTPYSLSTTTILANGKAMQGVFEYYKSVTFVSNCGSHPSTTSKGSPINTQYVF

SEQ ID NO: 7 - protein, 512 aa, Artificial Sequence
  NY-ESO-1 single chain trimer having a GGGAS linker at a Linker 1 position with a {G4A} substitution and a {Y84A} substitution
SLLMWITQVGGGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQWAAVVVPSGQEQRYTCHVQHEGLPKPLTLRWEPSSGSDYKDDDDKGGGGSGGGGSGGGGSQELTTICEQIPSPTLESTPYSLSTTTILANGKAMQGVFEYYKSVTFVSNCGSHPSTTSKGSPINTQYVF

SEQ ID NO: 8 - protein, 498 aa, Artificial Sequence
  NY-ESO-1 single chain trimer in the absence of a Linker 1
  MISC_FEATURE: annotated sequence of NY-ESO-1 peptide-MHC
SLLMWITQVIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSGSDYKDDDDKGGGGSGGGGSGGGGSQELTTICEQIPSPTLESTPYSLSTTTILANGKAMQGVFEYYKSVTFVSNCGSHPSTTSKGSPINTQYVF

SEQ ID NO: 9 - protein, 500 aa, Artificial Sequence
  NY-ESO-1 single chain trimer having two amino acid residues on the C-terminal region of the NY-ESO-1 peptide and a {Y84C} substitution in the absence of a Linker 1
SLLMWITQVGCIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSGSDYKDDDDKGGGGSGGGGSGGGGSQELTTICEQIPSPTLESTPYSLSTTTILANGKAMQGVFEYYKSVTFVSNCGSHPSTTSKGSPINTQYVF

SEQ ID NO: 10 - DNA, 51 bp, Saccharomyces cerevisiae
  misc_feature: PHO5
atgttcaagtccgtcgtctactctattttggctgcttctttggctaatgcc

SEQ ID NO: 11 - DNA, 57 bp, Saccharomyces cerevisiae
  misc_feature: SUC2
atgctgctgcaggccttcttgtttttgttggctggttttgctgctaagatttccgct

SEQ ID NO: 12 - DNA, 255 bp, Artificial Sequence
  app8
atgaggttcccctccattttcactgctgttttgtttgctgcttcttctgctttggctgctccagctaacactactactgaagatgagactgctcaaattccagctgaagctgttattgattactccgatttggaaggtgattttgatgctgctgctttgccattgtctaactctacaaacaatggtctgtcctctaccaataccaccattgcttctattgctgctaaagaagaaggcgttcaattggacaagaga

SEQ ID NO: 13 - DNA, 261 bp, Artificial Sequence
  app8 EA
atgaggttcccctccattttcactgctgttttgtttgctgcttcttctgctttggctgctccagctaacactactactgaagatgagactgctcaaattccagctgaagctgttattgattactccgatttggaaggtgattttgatgctgctgctttgccattgtctaactctacaaacaatggtctgtcctctaccaataccaccattgcttctattgctgctaaagaagaaggcgttcaattggataagagagaagct

SEQ ID NO: 14 - DNA, 114 bp, Artificial Sequence
  syn
atgaaggtcctgatcgtcttgttggctatttttgctgctttgccattggctttggctcaaccagttatttctactactgttggttctgctgctgaaggttctttggataagaga

SEQ ID NO: 15 - DNA, 120 bp, Artificial Sequence
  syn EA
atgaaggtcctgatcgtcttgttggctatttttgctgctttgccattggctttggctcaaccagttatttctactactgttggttctgctgctgaaggttctttggataagagagaagct

SEQ ID NO: 16 - DNA, 255 bp, Artificial Sequence
  appWT
atgagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtacagctggataaaaga

SEQ ID NO: 17 - DNA, 261 bp, Artificial Sequence
  appWT EA
atgagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtacagctggataaaagagaagct

SEQ ID NO: 18 - protein, 17 aa, Saccharomyces cerevisiae
  MISC_FEATURE: PHO5
MFKSVVYSILAASLANA

SEQ ID NO: 19 - protein, 19 aa, Saccharomyces cerevisiae
  MISC_FEATURE: SUC2
MLLQAFLFLLAGFAAKISA

SEQ ID NO: 20 - protein, 85 aa, Artificial Sequence
  app8
MRFPSIFTAVLFAASSALAAPANTTTEDETAQIPAEAVIDYSDLEGDFDAAALPLSNSTNNGLSSTNTTIASIAAKEEGVQLDKR

SEQ ID NO: 21 - protein, 87 aa, Artificial Sequence
  app8 EA
MRFPSIFTAVLFAASSALAAPANTTTEDETAQIPAEAVIDYSDLEGDFDAAALPLSNSTNNGLSSTNTTIASIAAKEEGVQLDKREA

SEQ ID NO: 22 - protein, 38 aa, Artificial Sequence
  syn
MKVLIVLLAIFAALPLALAQPVISTTVGSAAEGSLDKR

SEQ ID NO: 23 - protein, 40 aa, Artificial Sequence
  syn EA
MKVLIVLLAIFAALPLALAQPVISTTVGSAAEGSLDKREA

SEQ ID NO: 24 - protein, 85 aa, Artificial Sequence
  appWT
MRFPSIFTAVLFAASSALAAPVNTTTEDETAQIPAEAVIGYLDLEGDFDVAVLPFSNSTNNGLLFINTTIASIAAKEEGVQLDKR

SEQ ID NO: 25 - protein, 87 aa, Artificial Sequence
  appWT EA
MRFPSIFTAVLFAASSALAAPVNTTTEDETAQIPAEAVIGYLDLEGDFDVAVLPFSNSTNNGLLFINTTIASIAAKEEGVQLDKREA